(19) United States
(12) Patent Application Publication
(10) Pub. No.: US 2022/0204901 A1
(43) Pub. Date: Jun. 30, 2022

(54) Cell Culture System

(71) Applicant: Saint-Gobain Performance Plastics Corporation, Solon, OH (US)

(72) Inventors: Katie Campbell, Northborough, MA (US); Natalie Fekete, Marlborough, MA (US); Lei Zhang, Shrewsbury, MA (US); Rachel Z. Pytel, Newton, MA (US); Robert Gordon Pleydon, Worcester, MA (US)

(21) Appl. No.: 17/564,949

(22) Filed: Dec. 29, 2021

Related U.S. Application Data
(60) Provisional application No. 63/132,291, filed on Dec. 30, 2020.

(51) Int. Cl. C12M 1/00; C12M 1/04; C12M 1/12

(52) U.S. Cl. CPC C12M 23/14; C12M 23/24; C12M 29/10; C12M 25/14; C12M 23/26

(57) ABSTRACT

The disclosure relates generally to a cell culture apparatus and a cell culture method. One aspect of the disclosure is an oxygen-permeable bag comprising one or more polymer films defining a boundary of an interior compartment of the bag, each of the one or more polymer films comprising an inner layer adjacent the interior compartment of the bag, the inner layer comprising a fluoropolymer, and adhered to the inner layer, an outer layer comprising polymer; a first port formed in an exterior surface of the bag and in fluid communication with the interior compartment; a second port formed in an exterior surface of the bag and in fluid communication with the interior compartment; and contained in the interior compartment, a plurality of microcarriers.

### CROSS-REFERENCE TO RELATED APPLICATIONS

This application claims the benefit of priority of U.S. Provisional Patent Application No. 63/132,291, filed Dec. 30, 2020, which is hereby incorporated herein by reference in its entirety.

### BACKGROUND OF THE DISCLOSURE

### Field of the Disclosure

The disclosure relates generally to a cell culture apparatus and a cell culture method.

### Technical Background

Cell culture and cell isolation are important processes in a number of applications. For example, certain cells for use in therapeutic applications (e.g., immunotherapy, regenerative medicine, etc.) are typically isolated and cultured in vitro. For example, cells such as progenitor cells and mesenchymal stem cells, and monocytes and other immune cells are present in blood in relatively low concentrations, and accordingly are typically isolated from blood and cultured in vitro. Similarly, neuronal cells, cardiomyocytes, epithelial cells, and other cells for regenerative medicine (e.g., bone repair, skin repair, pancreatic islets regeneration, etc.) can be cultured in vitro.

Commercially available cell culture devices in the form of bags are a conventional format used for cell culture. Cell culture bags have the advantage of being disposable, which reduces preparation and clean up time. Additionally, cell culture bags are pre-sterilizable, inexpensive, easy to use and require minimal space for storage and use. Disposables also help reduce the risk of contamination for the cell culture and for the environment.

However, the surfaces of such bags are typically poor adhesion substrates for anchorage-dependent cells, which require an environment comparable to their natural cell niche to survive. Moreover, the interior surface area of such bags is relatively limited (e.g., compared to total bag volume).

Accordingly, there remains a need for cell culture and isolation articles that facilitate improved culturing of anchorage-dependent cells.

### SUMMARY OF THE DISCLOSURE

One aspect of the disclosure is an oxygen-permeable bag comprising one or more polymer films having edges bonded to form an interior compartment of the bag, each of the one or more polymer films comprising an inner layer adjacent the interior compartment of the bag, the inner layer comprising a fluoropolymer, and adhered to the inner layer, an outer layer comprising a polymer; a first port formed in an exterior surface of the bag and in fluid communication with the interior compartment; a second port formed in an exterior surface of the bag and in fluid communication with the interior compartment; and contained in the interior compartment, a plurality of microcarriers.

Another aspect of the disclosure is a bag (e.g., according to any embodiment as described herein) comprising an undivided interior compartment; a first liquid-permeable tube extending into and in fluid communication with the interior compartment, the first liquid-permeable tube being operatively coupled to the first port; and a second liquid-permeable tube extending into and in fluid communication with the interior compartment, the second liquid-permeable tube being operatively coupled to the second port; wherein the first liquid-permeable tube comprises a first inner support structure defining a central lumen of the tube and a first outer filter layer surrounding the first inner support structure.

Another aspect of the disclosure is a bag (e.g., according to any embodiment as described herein) comprising an undivided interior compartment; a first liquid-permeable tube extending into and in fluid communication with the interior compartment of the bag, the first liquid-permeable tube being operatively coupled to the port; and a second liquid-permeable tube extending into and in fluid communication with the interior compartment of the bag, the second liquid-permeable tube being operatively coupled to the second port; wherein the first liquid-permeable tube has an average pore size of 50-200 μm (e.g., 50-150 μm, or 75-150 μm).

Another aspect of the disclosure is a bag (e.g., according to any embodiment as described herein) comprising a porous membrane separating a first portion of the interior compartment from a second portion of the interior compartment, the second portion containing the plurality of microcarriers; and a third port formed in an exterior surface of the bag; wherein the first port and second port are each in fluid communication with the first portion of the interior compartment, and the third port is in fluid communication with the second portion of the interior compartment.

Another aspect of the disclosure is a bag (e.g., according to any embodiment as described herein) comprising a first porous membrane separating a first lateral portion of the interior compartment from a central portion of the interior compartment; a second porous membrane separating a second lateral portion of the interior compartment from the central portion of the interior compartment, the central portion containing the plurality of microcarriers; and a third port formed in an exterior surface of the bag; wherein the first port is in fluid communication with the first lateral portion of the interior compartment, the second port is in fluid communication with the second lateral portion of the interior compartment, and the third port is in fluid common cation with the central portion of the interior compartment.

Another aspect of the disclosure is a method for cultivating cells, comprising adding anchorage-dependent cells and media to a bag (e.g., according to any embodiment as described herein).

### BRIEF DESCRIPTION OF THE DRAWINGS

FIG. 1 is a schematic top-down view (top) and cross-sectional view (bottom) of a bag according to one embodiment of the disclosure.

FIG. 2 is a partial cross-sectional view of a polymer film suitable for use in the construction of the bags as described herein.

FIG. 3 is a schematic cutaway view of a liquid-permeable tube according to one embodiment of the disclosure.

FIG. 4 is a schematic cutaway view of a liquid-permeable tube according to one embodiment of the disclosure.

FIG. 5 is a schematic top-down (top) and cross-sectional (bottom) view of a bag according to one embodiment of the disclosure.

FIG. 6 is a schematic cross-sectional view of a bag according to one embodiment of the disclosure.

FIG. 7 is a schematic cross-sectional view of a bag according to one embodiment of the disclosure.

FIG. 8 is a schematic top-down view of a bag according to one embodiment of the disclosure.

FIG. 9 is a picture of a comparative, commercially available fluoropolymer bag after treatment in an autoclave (see Example 1).

FIG. 10 is a picture of an oxygen-permeable bag according to one embodiment of the disclosure, after treatment in an autoclave (see Example 1).

### DETAILED DESCRIPTION

The disclosure relates to articles and methods for culturing and isolating anchorage-dependent cells in a cell culture bag. The present inventors note that conventional cell culture bags—the surfaces of which are typically unsuitable substrates for anchorage-dependent cells—can moreover be unsuitable for microcarrier cell culture. As the person of ordinary skill in the art will appreciate, a “microcarrier” cell culture includes discrete micrometer-scale particles (e.g., spheroidal “microcarriers,” or “microbeads”), the surfaces of which can function as an adhesion substrate for anchorage-dependent cells.

The present inventors note that the interior surfaces of a cell culture bag can be distorted by mechanical stress resulting from, for example, bag preparation (e.g., structural changes effected by sterilization) or use (e.g., unevenly applied force from a bag support, or a bag-rocking device). The macro-scale features on the bag surface that result from such distortion can cause localization, or “pooling,” of microcarriers during a cell culture process, resulting in an undesirable, uneven distribution of microcarriers throughout the bag volume. Such features can also significantly increase the amount of shear stress and collision stress to which carrier-anchored cells are subjected (e.g., during stirring, or rocking), both directly (i.e., collisions between macro-scale surface features and microcarriers) and indirectly (i.e., increased collisions between microcarriers in “pooled” areas of an unevenly distributed microcarrier population).

Advantageously, the present inventors have determined that the interior surfaces of culture bags formed from an oxygen-permeable polymer film comprising an inner fluoropolymer layer adhered to an outer polymer layer can desirably remain relatively undistorted throughout preparation (e.g., sterilization in an autoclave) and use (e.g., incubation on a rocking device). Accordingly, microcarriers contained in the bag during a cell culture process can remain relatively evenly distributed, and exposure of anchored cells to shear forces and collision forces can be significantly reduced. The present inventors have moreover determined that such bags can advantageously include one or more perfusive components that facilitate minimally disruptive removal and replenishment of feed media during cell culture and/or efficient recovery of cultivated cells.

Accordingly, one aspect of the disclosure is an oxygen-permeable bag comprising one or more polymer films (e.g., a first polymer film and a second polymer film) defining a boundary of an interior compartment of the bag (e.g., by having edges bonded to form the bag). Each of the polymer films comprises an inner layer adjacent the interior compartment of the bag, the inner layer comprising a fluoropolymer, and, adhered to the inner layer, an outer layer comprising a polymer. The bag includes a first port formed in an exterior surface of the bag and in fluid communication with the interior compartment, and a second port formed in an exterior surface of the bag and in fluid communication with the interior compartment. And the bag contains, in the interior compartment, a plurality of microcarriers.

As noted above, in various embodiments the oxygen-permeable bag can be in the form of an oxygen-permeable bag comprising one or more polymer films having edges bonded to form an interior compartment of the bag, each of the one or more polymer films comprising an inner layer adjacent the interior compartment of the bag, the inner layer comprising a fluoropolymer, and adhered to the inner layer, an outer layer comprising a polymer; a first port formed in an exterior surface of the bag and in fluid communication with the interior compartment; a second port formed in an exterior surface of the bag and in fluid communication with the interior compartment; and contained in the interior compartment, a plurality of microcarriers.

In various embodiments as otherwise described herein, the one or more polymer films include a first polymer film and a second polymer film, having edges bonded together to form edges around an interior compartment of a bag. However, in other embodiments, a single polymer film can be folded over and bonded to itself to form a bag, or a tubular film can be welded on two edges to form a bag. In any case, when two separate films are used, only one polymer film need have the laminate structure described herein; the other film may also have the laminate structure, or it may have some different structure.

As described above, each of the polymer films (i.e., the laminate polymer films, recognizing that only one such film need be present in the bag) comprises an inner layer comprising a fluoropolymer. In various embodiments as otherwise described herein, the inner layer comprises substantially fluoropolymer, e.g., at least 80 wt. %, or at least 85 wt. %, or at least 90 wt. %, or at least 95 wt. %, or at least 97.5 wt. %, or at least 98 wt. %, or at least 99 wt. % fluoropolymer.

In various embodiments as otherwise described herein, the inner layer comprises one or more fluoropolymers selected from ethylene-propylene (FEP), polytetrafluoroethylene (PTFE), polyvinylidenefluoride (PVDF), tetrafluoroethylene-perfluoro(propyl vinyl ether) (PFA), polyvinylidene difluoride (PVF), polychlorotrifluoroethlylene (PCTFE), tetrafluoroethylene/hexafluoropropylene/ethylene copolymers (HTE), chlorotrifluoroethylene/vinylidenefluoride copolymers, chlorotrifluoroethylene/hexafluoropropylene copolymers, ethylene/chlorotrifluoroethylene copolymers (ECTFE), ethylene/trifluoroethylene copolymers, ethylene/tetrafluoroethylene copolymers (ETFE), tetrafluoroethylene/propylene copolymers (TFE/P), tetrafluoroethylene/hexafluoropropylene copolymers (FEP/HFP), hexafluoropropylene/tetrafluoroethylene/vinylidene copolymers (THV), and perfluoro(1-butenyl vinyl ether) homocyclopolymers having functionalized polymer-end groups. In certain desirable embodiments, the inner layer comprises fluorinated ethylene-propylene. For example, in certain such embodiments, the inner layer comprises at least 80 wt. % (e.g., at least 90 wt. %, or at least 95 wt. %) fluorinated ethylene-propylene.

In various embodiments, the inner layer comprises a fluoropolymer selected from amorphous, fluorine-comprising polymers that are non-crystalline (e.g., when measured by DSC), or have a heat of melting of less than 2 J/g. For example, in certain such embodiments, the inner layer comprises a copolymer of tetrafluoroethylene with functional or non-functional monomers such as fluoroolefins having 2-8 carbon atoms and fluorinated alkyl vinyl ether in which the alkyl group contains 1 or 3 to 5 carbon atoms. In various embodiments, the non-functional monomers include one or more of hexafluoropropylene (HFP), chlorotrifluoro ethylene (CTFE), PEVE, PMVE and perfluoro-(propylene vinyl ether) (PPVE). In various embodiments, the functional monomers include one or more of perfluoroethyl vinyl ether (EVE), CF₂═CFOCF₂CFCF₃OCF₂CF₂COOCH₃ (EVE-carbamate), CF₂═CFOCF₂CFCF₃OCF₂CF₂SO₂F (PSEPVE), CF₂═CFOCF₂CFCF₃OCF₂CF₂CN (8CNVE), N₃(CF₂═CFOCF₂CFCF₃OCF₂CF₂)₃ (EVE-triazine), CF₂═CFOCF₂CFCF₃OCF₂CF₂CN (EVE-CN), CF₂═CFOCF₂CFCF₃OCF₂CF₂CH₂OH (EVE-OH), CF₂═CFOCF₂CFCF₃OCF₂CF₂CH₂PO₂(OH)₂ (EVE-P) CF₂═CFOCF₂CFCF₃OCF₂CF₂CH₂COOH (EVE-COOH), and 2,2-bistrifluoromethyl-4,5-difluoro-1,3-dioxole (PDD).

In various embodiments as otherwise described herein, the inner layer can include one or more commercially available amorphous fluoropolymers, such as those from DuPont, Wilmington, Del.: TEFLON® SF60 (TFE/PMVE/PEVE, DuPont, Wilmington Del.), TEFLON® SF61 (TFE/PMVE/PEVE/EVE-P), TEFLON® SF50 (TFE/HFP), Teflon® AF 1600 (PDD/TFE), and TEFLON® AF2130 (PDD/CTFE); from Asahi Corporation of Tokyo, Japan: CYTOP® (CYTOP type A, CYTOP type M, CYTOP type S, or CYTOP NM); from MY Polymers Corporation of Rehovot, Israel (MY-133); or from Nusil Corporation of Carpinteria, Calif. (LS-233).

In various embodiments as otherwise described herein, the inner layer (e.g., comprising substantially fluorinated ethylene-propylene) has a thickness of 0.001-0.7 mm, e.g., 0.001-0.4 mm, or 0.001-0.1 mm, or 0.005-0.7 mm, or 0.005-0.4 mm, or 0.005-0.1 mm, or 0.01-0.7 mm, or 0.01-0.4 mm, or 0.01-0.1 mm. In certain desirable embodiments as otherwise described herein the inner layer comprises at least 95 wt. % fluorinated ethylene-propylene and has a thickness of 0.01-0.1 mm.

As described above, each of the polymer films (i.e., the laminate polymer films, recognizing that only one such film need be present in the bag) comprises an outer layer comprising a polymer. A variety of polymers can be used, and the person of ordinary skill in the art will select a desirable polymer based on the disclosure herein, especially with respect to gas permeability and wrinkliness. In certain desirable embodiments, the outer layer is substantially free of fluoropolymer, e.g., does not include more than 5% fluoropolymer, e.g., no more than 1% fluoropolymer.

In various embodiments as otherwise described herein, the outer layer comprises an elastomer. As the person of ordinary skill in the art will appreciate, “elastomers,” or “rubber” include polymers formed from monomers including carbon, hydrogen, oxygen and/or silicon with viscoelastic properties and relatively weak inter-molecular forces. Elastomers typically have a relatively low Young's modulus, and a relatively high failure strain. In various embodiments as otherwise described herein, the outer layer comprises substantially elastomer, e.g., at least 80 wt. %, or at least 85 wt. %, or at least 90 wt. %, or at least 95 wt. %, or at least 97.5 wt. %, or at least 98 wt. %, or at least 99 wt. % elastomer.

In various embodiments as otherwise described herein, the outer layer comprises one or more unsaturated rubbers cured by sulfur vulcanization, such as natural rubber (NR), synthetic polyisoprene rubber (IR), polybutadiene rubber (BR), chloroprene rubber (CR), butyl rubber (IIR), halogenated butyl rubbers (CIIR, BIIR), styrene-butadiene rubber (SBR), nitrile rubber (NBR) and hydrogenated nitrile rubber (HNBR). In various embodiments as otherwise described herein, the outer layer comprises one or more unsaturated runners uncurable by sulfur vulcanization, such as ethylene propylene rubber (EPM), ethylene propylene diene rubber (EPDM), epichlorohydrin rubber (ECO), polyacrylic rubber (ACM, ABR), silicone rubber (SI, Q, VMQ), fluorosilicone rubber (FSR, FVMQ), fluoroelastomers (FKM, FEPM), perfluoroelastomers (FFKM), polyether block amides (PEBA), chlorosulfonated polyethylene (CSM), thermoplastic urethanes (TPUs), including thermoplastic silicones, such as a GENIOMER®, cyclic olefin copolymers, polyolefin elastomers, elastomeric PET, and ethylene-vinyl acetate (EVA). In certain such embodiments, the one or more unsaturated rubbers are present in a combined amount of at least 80 wt. % (e.g., at least 90 wt. %, or at least 97.5 wt. %, or at least 99 wt. %) of the outer layer.

In various embodiments as otherwise described herein, the outer layer comprises one or more thermoplastic polyurethanes or thermoset polyurethanes. In certain such embodiments, the outer layer comprises one or more thermoplastic polyurethanes (TPUs), such as thermoplastic polyurethanes based upon methylene diisocyanate (MDI) or toluene diisocyanate (TDI), including both polyester and polyether grades of polyols. For example, in various embodiments, the outer layer comprises one or more thermoplastic polyurethanes based upon commercially available “pre-polymers” including “TEXIN”, a tradename of Bayer Materials Science, “ESTANE”, a tradename of Lubrizol, “PELLETHANE”, a tradename of Dow Chemical Co., and “ELASTOLLAN”, a tradename of BASF, Inc. In certain such embodiments, the one or more thermoplastic polyurethanes or thermoset polyurethanes are present in a combined amount of at least 80 wt. % (e.g., at least 90 wt. %, or at least 97.5 or at least 99 wt. %) of the outer layer.

In various embodiments as otherwise described herein, the outer layer comprises one or more thermoplastic silicones, such as GENIOMER® 140 Silicone TPE, GENIOMER® 200 Silicone TPE Elastomer (90% polydimethylsiloxane and isocyanate), GENIOMER®, 60 Silicone TPE, GENIOMER® 80 Silicone TPE or GENIOMER 145 TPE, all of which comprise 90% polydimethylsiloxane and isocyanate. In certain such embodiments, the one or more thermoplastic silicones are present in a combined amount of at least 80 wt. % (e.g., at least 90 wt. %, or at least 97.5 wt. %, or at least 99 wt. %) of the outer layer.

In certain desirable embodiments as otherwise described herein, the outer layer comprises polymethylpentene (PMP) (i.e., a thermoplastic polymer of methylpentene monomer units). For example, in certain such embodiments, the outer layer comprises at least 80 wt. % (e.g., at least 90 wt. %, or at least 95 wt. %) polymethylpentene.

In various embodiments as otherwise described herein, the outer layer comprises silicone rubber. For example, in certain such embodiments, the outer layer comprises at least 80 wt. % (e.g., at least 90 wt. %, or at least 95 wt. %) silicone rubber.

In certain such embodiments, the outer layer comprises silicone rubber including a silicone polymer matrix including polyalkylsiloxanes formed from, for example, one or more of dimethylsiloxane, diethylsiloxane, dipropylsiloxane, methylethylsiloxane, and methylpropylsiloxane. In certain desirable embodiments, the silicone rubber comprises a polydialkylsiloxane such as, for example, polydimethylsiloxane (PDMS). In certain such embodiments, the silicone polymer is relatively non-polar and substantially free of halide functional groups (e.g., Cl, Br) and phenyl functional groups.

In various embodiments as otherwise described herein, the outer layer comprises one or more polyorganosiloxanes selected from polydimethylsiloxanes which are endblocked by vinyldimethylsiloxy groups at both ends, dimethylsiloxane-vinylmethylsiloxane copolymers which are endblocked by vinyldimethylsiloxy groups at both ends, and dimethylsiloxane-methylphenylsiloxane copolymers which are endblocked by vinyldimethylsiloxy groups at both ends.

In various embodiments as otherwise described herein, the silicone rubber comprises a platinum catalyzed liquid silicone rubber (LSR) or a high consistency gum rubber (HCR). In various embodiments, the silicone rubber comprises a peroxide catalyzed silicone rubber (LSR) or a high consistency gum rubber (HCR), such as, for example, SILMEDIC®, a peroxide based silicone produced by Saint-Gobain. An example of HCR silicone rubber is GE 94506 HCR available from GE Plastics. And examples of LSR silicone rubber include Wacker 3003 by Wacker Silicone of Adrian, Mich. and Rhodia 4360 by Rhodia Silicones of Ventura, Calif.

In various embodiments as otherwise described herein, the outer layer (e.g., comprising substantially polymethylpentene or silicone rubber) has a thickness of 0.01-5 mm, e.g., 0.01-1 mm, or 0.01-0.5 mm, or 0.05-5 mm, or 0.05-1 mm, or 0.05-0.5 mm, or 0.1-5 mm, or 0.1-1 mm, or 0.1-0.5 mm. In certain desirable embodiments as otherwise described herein, the outer layer comprises a least 95 wt. % polymethylpentene and has a thickness of 0.1-0.5 mm. In other desirable embodiments as otherwise described herein, the outer layer comprises at least 95 wt. % silicone rubber and has a thickness of 0.1-0.5 mm.

In certain desirable embodiments as otherwise described herein, the inner layer has a thickness of 0.001-0.7 mm, and the outer layer has a thickness of 0.01-5 mm. For example, in various embodiments, the inner layer has a thickness of 0.01-0.4 mm, and the outer layer has a thickness of 0.05-1 mm. In another example, in various embodiments, the inner layer has a thickness of 0.01-0.1 mm, and the outer layer has a thickness of 0.1-0.5 mm. In certain such embodiments, the inner layer comprises substantially (e.g., at least 95 wt. %) fluorinated ethylene-propylene. In certain such embodiments, the outer layer comprises substantially (e.g., at least 95 wt. %) silicone rubber.

As described above, the inner layer and outer layer of the polymer films are adhered together. In various embodiments, the adhered layers are formed by a coating process. In certain such embodiments, the adhered inner layer and outer layer are the product of dispersing a castable fluoropolymer composition onto a polymer layer (e.g., an elastomer layer). Of course, in other such embodiments, the adhered layers are the product of dispersing a castable polymer composition (e.g., a castable elastomer composition) onto a fluoropolymer layer. The person of ordinary skill in the art will appreciate that a “castable” polymer (e.g., fluoropolymer or non-fluoropolymer) is capable of being dispersed, dissolved, suspended, emulsified or otherwise distributed in a liquid carrier medium to provide a polymer composition that can be deposited onto a supporting material to form a polymer layer. Of course, “castable” polymers can also include those polymers that can be melted or other otherwise processed in a liquid state, and then cooled or otherwise cured (e.g., by UV, IR, initiators, or ebeam) to form a solid polymer layer. Suitable carrier liquids (e.g., DMAC, NMP, glycol ethers, water) and methods for casting the polymers described herein are known in the art.

In various embodiments as otherwise described herein, the adhered layers are formed by a coating process involving chemical bonding. For example, in various embodiments as otherwise described herein, the outer layer comprises substantially (e.g., at least 80 wt. %, at least 90 wt. %, or at least 95 wt. %) silicone rubber, and the inner fluoropolymer layer adhered thereto is the product of casting a fluoropolymer composition containing a reactive compound capable of bonding to silicone rubber. In certain such embodiments, the reactive compound is selected from hydrocarbon silicon-containing compounds such as, for example, compounds having a siloxane bond, silane coupling agents having an alkoxysilyl group, functional silanes having a chlorosilyl group or silazane, amino-silane, and silylating agents. Of these, compounds having a siloxane bond, and silane coupling agents having an alkoxysilyl group, are preferred. In various embodiments, the reactive compound has a siloxane bond (e.g., end-modified dimethylsiloxanes, condensation-type or addition-type liquid silicones, silicate salts, and acrylic silicone polymers).

For example, in various embodiments as otherwise described herein, the outer layer comprises substantially (e.g., at least 80 wt. %, at least 90 wt. %, or at least 95 wt. %) silicone rubber, and the inner fluoropolymer layer adhered thereto is the product of casting a composition including a fluoropolymer having an amino-silane end modification (e.g., CYTOP type M) or a fluoropolymer having a carboxyl group end modification (e.g., CYTOP type A), and optionally including a coupling agent. In such embodiments, adherence by chemical bonding can occur by one or more of condensation, alkylation, amidation, silylation, etherification (e.g., directly or through a coupling agent). For example, in certain such embodiments, the inner fluoropolymer layer is the product of casting a composition including an isocyanate compound that reacts with hydroxyl, amino, or sulfur groups. In other embodiments, the fluoropolymer layer is adhered to the outer layer through hydrogen bonding.

In other embodiments as otherwise described herein, the inner layer and outer layer are bonded by one or more of chemical bonding, adhesive bonding, thermal fusion bonding, solvent bonding, laser welding, surface treatment, extrusion, co-extrusion, and lamination. For example, in certain desirable embodiments, the inner layer (e.g., comprising substantially fluorinated ethylene propylene) and the outer layer (e.g., comprising substantially silicone rubber) are bonded by one or more surface treatments selected from C-treatment (Saint-Gobain Performance Plastics Corporation, U.S. Pat. No. 6,726,979), corona discharge, plasma treatment, and etching, In other embodiments, the inner layer and the outer layer are bonded by a chemical treatment involving additives or primers that can be used alone or in conjunction with the treatment methods described herein.

For example, in certain desirable embodiments as otherwise described herein, the inner layer comprises substantially (e.g., at least 80 wt. %, at least 90 wt. %, or at least 95 wt. %) fluorinated ethylene-propylene (e.g., C-treated fluorinated ethylene-propylene), and the outer layer adhered thereto is the product of priming and coating the fluorinated ethylene-propylene with liquid silicone rubber (LSR) silicone (e.g., by extrusion). In other embodiments, the inner layer and outer layer are pre-laminated together (e.g., similarly to that for LIGHTSWITCH® Complete product (Saint-Gobain Performance Plastics Corporation, Valley Forge, Pa.)).

The present inventors note that the inner layer of the bags described herein can desirably have a low total organic carbon (TOC). The person of ordinary skill in the art will appreciate that “total organic carbon” is the amount of carbon bound in an organic compound and is often used as a non-specific indicator of pharmaceutical manufacturing equipment, among other things. Total organic carbon is utilized as a process control attribute in the biotechnology industry to monitor the performance of unit operations that employ purification and distribution systems.

Total organic carbon is measured by extraction from an internal surface area of a bag or the surface area of another item (with results provided in units of mg/cm², the total extractable organic carbon per square centimeter of the surface area). A material is extracted by being contacted by a given volume of purified water at 70° C. for 24 hours.

One example of the total organic carbon value of a fluorinated ethylene propylene bag (i.e., using FEP films) is <0.01 mg/cm². For silicone tubing, extraction ratios may be 14.6 cm²/mL (e.g., Biosil) or may be 15.9 cm²/mL (e.g., SR139), and one example of the total organic carbon value of a silicone Biosil tube is 0.021 mg/cm², and one example of the total organic carbon value of silicone SR139 tubing is 0.008 mg/cm². For at least certain silicone tubing, the samples may be diluted, as the volume and concentration of the extraction cause the value to be above the maximum detection of the machine. The dilution and different extraction ratio requires the comparison of these samples with bag samples to provide a weight/area value.

As quantified herein, total organic carbon is measured according to US Pharmacopeia (USP) 643 using equipment that utilizes a high temperature wet oxidation reaction of UV-promoted chemical oxidation (Ultra-Clean Technology Handbook: Volume 1: Ultra-Pure Water, Ohmi, Tadahiro; CRC Press, 1993, pp. 497-517). Purified water is placed in contact with the polymer for 24 hours at 70° C., at a ratio of 3 cm² of article surface area to 1 mL of water. The water is removed from contact with the polymer and tested in a TOC analyzer. A suitable piece of equipment is a TEKMAR DOHRMANN Model Phoenix 8000 TOC analyzer.

Accordingly, in various embodiments as otherwise described herein, one or more of (e.g., each of) the polymer films making up the bag are gas-permeable and comprise an inner layer having a total organic carbon in water of less than 0.1 mg/cm², e.g., or less than 0.5 mg/cm², or less than 0.1 mg/cm², or less than 0.01 mg/cm², or less than 0.001 mg/cm². For example, in certain such embodiments, each of the polymer films comprise an inner layer having a total organic carbon in water of 0.001 mg/cm² to 0.1 mg/cm², e.g., 0.001 mg/cm² to 0.075 mg/cm², or 0.001 mg/cm² to 0.05 mg/cm2, or 0.001 mg/cm² to 0.01 mg/cm².

In certain desirable embodiments as otherwise described herein, the outer layer comprises substantially (e.g., at least 80 wt. %, at least 90 wt. %, or at least 95 wt. %) silicone rubber, the inner layer comprises substantially (e.g., at least 80 wt. %, at least 90 wt. %, or at least 95 wt. %) fluorinated ethylene propylene, and the inner layer has a total organic carbon in water of 0.001 mg/cm² to 0.05 mg/cm², e.g., 0.001 mg/cm² to 0.01 mg/cm².

As described above, the bag is oxygen-permeable. The present inventors note that the oxygen-permeable polymer films described herein can facilitate the gas exchange necessary for cell culture (e.g., replenishment of oxygen), reducing or even eliminating the need for one or more additional gas feeds to or from the bag. Accordingly, in various embodiments as otherwise described herein, at least one of (e.g., each of) the polymer films making up the bag has a gas permeability (e.g., oxygen permeability) of at least 100 cc/m² per day, e.g., at least 500 cc/m² per day, or at least 1,000 cc/m² per day, or at least 2,000 cc/m² per day. As quantified herein, oxygen permeability is measured with a MOCON Ox-tran 2/21H Oxygen Analyzer, following ASTM D3985, at 25° C. In another aspect of film permeability, normalized units (cc-mm/m²-day) can be used to show a film of any thickness. For example, the converted range for a 5 mm film would be from about 12.7 cc-mm/m²-day to at least about 279 cc-mm/m²-day at a temperature of 25° C. The permeability of the construct/composite can be expressed in cc/m² terms, as it would be comprised of two layers.

The present inventors moreover note that the bag as otherwise described herein can desirably be permeable to carbon dioxide (e.g., to facilitate removal of accumulated carbon dioxide). Accordingly, in certain desirable embodiments as otherwise described herein, at least one of (e.g., each of) the polymer films making up the bag has a carbon dioxide permeability of at least 5,000 cc/m² per day, e.g., at least 10,000 cc/m² per day, or at least 15,000 cc/m² per day, or at least 20,000 cc/m² per day. For example, in various embodiments as otherwise described herein, at least one of (e.g., each of) the polymer films making up the bag has a carbon dioxide permeability of 10,000-40,000 cc/m² per day, e.g., 15,000-35,000 cc/m² per day, or 20,000-30,000 cc/m² per day. As quantified herein, carbon dioxide permeability is measured with a MOCON Permatran-C 441 CO2TR Analyzer, following ASTM F2476, at 25° C., 100% CO₂, and 0% relative humidity on both sides of the film.

In certain desirable embodiments as otherwise described herein, the outer layer comprises substantially (e.g., at least 80 wt. %, at least 90 wt. %, or at least 95 wt. %) silicone rubber, the inner layer comprises substantially (e.g., at least 80 wt. %, at least 90 wt. %, or at least 95 wt. %) fluorinated ethylene propylene, and at least one of (e.g., each of) the polymer films has a gas permeability in the range of 1000-25,000 cc/m² per day and a total organic carbon in water of less than 0.1 mg/cm². In certain such embodiments, each of the polymer films comprise an inner layer having a total organic carbon in water of 0.001 mg/cm² to 0.1 mg/cm², e.g., 0.001 mg/cm² to 0.075 mg/cm², or 0.001 mg/cm² to 0.05 mg/cm2, or 0.001 mg/cm² to 0.01 mg/cm².

As described above, the bag contains, in the interior compartment, a plurality of microcarriers. In certain desirable embodiments, the microcarriers contained in the interior compartment of the bag are porous and non-degradable (e.g., in aqueous cell culture media). For example, in various embodiments as otherwise described herein, the microcarriers comprise polystyrene, cross-linked dextran, or cellulose. In certain desirable embodiments, the microcarrier surface comprises positively or negatively charged functional groups (e.g., covalently attached to the polystyrene, cross-linked dextran, cellulose, etc.). For example, in certain such embodiments, the microcarriers are substantially polystyrene, and have a net-negatively charged surface comprising oxygen-containing functional groups (e.g., covalently attached to the polystyrene). The person of ordinary skill in the art will appreciate that such “surface-treated” microcarriers can have a relatively hydrophilic surface that facilitates cell attachment and/or spreading.

In various embodiments as otherwise described herein, the microcarriers comprise one or more extracellular matrix compounds, e.g., collagen I, poly-L-lysine, fibronectin, retronectin, hyaluronic acid, polydopamine. For example, in certain such embodiments, the microcarriers comprise one or more extracellular matrix compounds (e.g., collagen or polydopamine) covalently attached to the surface of a core particle comprising substantially polystyrene, cross-linked dextran, or cellulose. In other embodiments as described herein, the microcarriers comprise at least at least 90 wt. %, e.g. at least 95 wt. %, or at least 97.5 wt. %, or at least 98 wt. %, or at least 99 wt. %, or at least 99.5 wt. % of one or more of polystyrene, cross-linked dextran, or cellulose.

In various embodiments as otherwise described herein, the microcarriers have an average diameter of 100-400 μm, e.g., 100-300 μm, or 100-275 μm, or 110-400 μm, or 110-300 μm, or 110-275 μm, or 120-400 μm, or 120-300 μm, or 120-275 μm. In various embodiments as otherwise described herein, the microcarriers have an average density of 1-1.25 g/mL, e.g., 1-1.15 g/mL, or 1-1.1 g/mL. In various embodiments as otherwise described herein, the microcarriers have an average specific surface area of 0.1-10 cm²/mg, e.g., 0.1-7.5 cm²/mg, or 0.5-10 cm²/mg, or 0.5-7.5 cm²/mg, or 1-10 cm²/mg, or 1-7.5 cm²/mg.

In various embodiments as otherwise described herein, the ratio of the combined mass of the plurality of microcarriers contained in the interior compartment (i.e., in grams) to the volume of the interior compartment (i.e., in liters) is 1-15 g/L, e.g., 1-12.5 g/L, or 1-10 g/L, or 2.5-15 g/L, or 2.5-12.5 g/L, or 2.5-10 g/L, or 5-15 g/L, or 5-12.5 g/L, or 5-10 g/L. In various embodiments as otherwise described herein, the microcarriers have an average specific surface area of 0.1-10 cm²/mg (e.g., 0.5-10 cm²/mg), and the ratio of the combined mass of the plurality of microcarriers contained in the interior compartment to the volume of the interior compartment is 1-15 g/L (e.g., 2.5-10 g/L).

In various embodiments as otherwise described herein, the plurality of microcarriers contained in the interior compartment has a total surface area that is at least 100%, e.g., at least 500%, or at least 1,000%, or at least 5,000%, or at least 10,000%, of a total surface area of the inner layer adjacent the interior compartment of the bag.

As described above, the bag described herein can advantageously limit uneven microcarrier distribution and/or exposure of anchored cells to undesirable shear and collision forces during cell culture. Accordingly, another aspect of the disclosure is a method for cultivating cells, comprising adding anchorage-dependent cells and culture media to a bag described herein. In various embodiments as otherwise described herein, the cells comprise blood cells or immune cells. In various embodiments as otherwise described herein, the cells are stem cell, multipotent stromal cells, hepatocytes, keratinocytes, endothelial epithelial cells, neurons. In various embodiments as otherwise described herein, the cells are differentiated stem cells, such as, for example, chondrocyte-like, osteoblast-like, or adipocyte-like differentiated stem cells. In various embodiments as otherwise described herein, the cells are endothelial progenitor cells, mesenchymal stromal cells, or loosely adherent cells such as, for example, monocytes.

In various embodiments as otherwise described herein, a number of anchorage-dependent cells is added to the bag to provide a ratio of cells to a total surface area of the plurality of microcarriers of 1,000-10,000 cells/cm², e.g., 1,000-9,000 cells/cm², or 1,000-7,500 cells/cm², or 2,500-10,000 cells/cm², or 2,500-9,000 cells/cm², or 2,500-7,500 cells/cm², or 3,500-10,000 cells/cm², or 3,500-9,000 cells/cm², or 3,500-7,500 cells/cm².

The bag can be maintained at a desired incubation temperature, and can be agitated. For example, in various embodiments as otherwise described herein, the method includes rocking the bag.

In various embodiments as otherwise described herein, the method further comprises separating cultivated cells from the microcarriers (e.g., by exposing anchored cells to trypsin), and then removing the cultivated cells from the bag (e.g., through a fitter configured to retain the microcarriers in the interior compartment of the bag).

As described above, the bags described herein can advantageously include one or more perfusive components. For example, in various embodiments as otherwise described herein, the bag comprises an undivided interior compartment, a first liquid-permeable tube extended into and in fluid communication with the interior compartment, the first liquid-permeable tube being operatively coupled to the port, and a second liquid-permeable tube extended into and in fluid communication with the interior compartment, the second liquid-permeable tube being operatively coupled to the second port. In such embodiments, the first liquid-permeable tube comprises a first inner support structure defining a central lumen of the tube and a first outer filter layer surrounding the first inner support structure. The present inventors note that, desirably, feed media can be perfused through the interior compartment of the bag without appreciably disturbing anchored cells and/or diminishing the number of contained microcarriers by flowing media from the second tube, through the interior volume, and out of the first tube. The first filter layer of the first tube can prevent microcarriers from escaping the interior volume of the interior compartment. Anchorage-dependent cells can thus be grown and concentrated in the interior compartment without feed media supply limitations and/or waste removal limitations. When present, the third port can be used for media sampling and introduction of cells and/or microcarriers into the interior compartment. The present inventors moreover note that, advantageously, following a cell culture process, cultivated cells can be detached from the microcarriers and removed from the bag through the first filter layer (e.g., which retains the microcarriers in the interior compartment).

But the person of ordinary skill in the art can arrange a filter with respect to one or more of the first and second port in a variety of ways, i.e., suitable to filter media exiting the internal compartment therethrough to, e.g., prevent microcarriers from leaving the bag during perfusion.

Notably, in such embodiments, the interior compartment can be undivided (e.g., there is no membrane or other porous structure that divides the interior compartment such that the first and second tubes are in a first sub-compartment and a third port is connected to a second sub-compartment divided from the first sub-compartment by the membrane or other porous structure). The present inventors note that this can advantageously allow for good fluid transfer between the incubating cells and the perfused media.

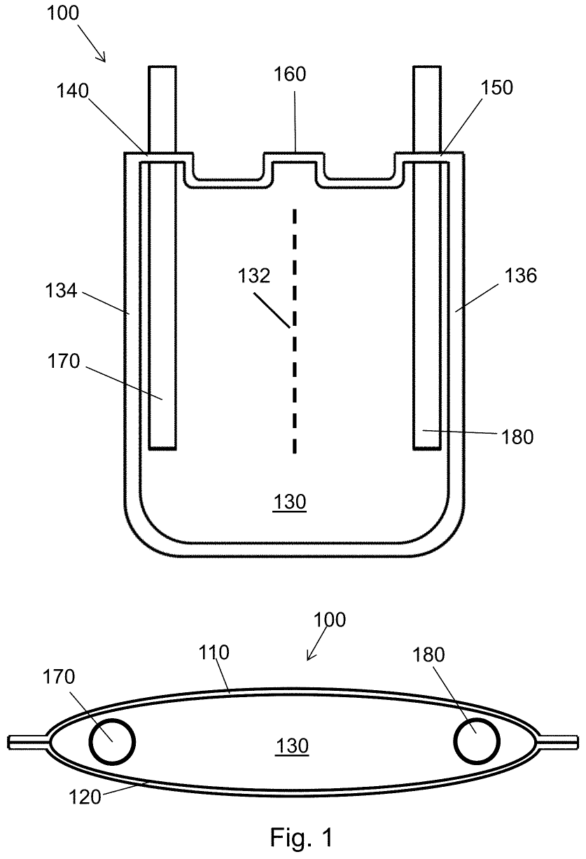

An embodiment of such a bag is shown in schematic top-down view (top) and cross-sectional view (bottom) in FIG. 1. Bag 100 of FIG. 1 includes a first polymer film 110 and a second polymer film 120, bonded together at edges thereof to form edges around an undivided interior compartment 130 having a major axis 132. Bag 100 further includes first port 140 and a second port 150, both in fluid communication with the interior compartment 130, In the particular embodiment of FIG. 1, first port 140 is operatively coupled to a first liquid-permeable tube 170, and second port 150 is operatively coupled to a second liquid-permeable tube 180. In the embodiment of FIG. 1, Bag 100 further includes third port 160 in fluid communication with interior compartment 130; however, the person of ordinary skill in the art will appreciate that the third port need not be present in all embodiments contemplated herein. Microcarriers 135 can be disposed in the interior compartment 130.

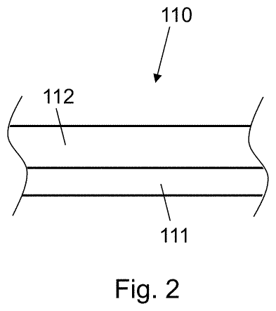

FIG. 2 is a partial cross-sectional view of a polymer film suitable for use in the construction of the bags as described herein, e.g., the bag of FIG. 1. Here, polymer film 110 has an inner layer 111 that comprises (e.g., is) a fluoropolymer as otherwise described herein, and an outer layer 112 that comprises (e.g., is) a polymer (e.g., a non-fluorinated polymer) as otherwise described herein.

The bags described herein can be provided in a variety of volumes. Though not particularly limited, the volume of interior compartment can be, for example, in the range of 5 mL to 3500 mL, e.g., from 10 mL to 500 mL, or from 10 mL to 100 mL, or from 100 mL to 3000 mL, or from 500 mL to 2500 mL, or from 500 mL to 2000 mL. But as the person of ordinary skill in the art would appreciate, bags of different volumes are also contemplated.

The bags described herein are desirably configured to allow perfusion of media through a substantial portion of the interior volume by flowing between the first port and the second port.

For example, in various desirable embodiments of bags that include a first tube and a second tube, like that of FIG. 1 described above, the first tube is disposed adjacent a first lateral edge of the bag, and the second tube is disposed adjacent a second, opposed, lateral edge of the bag; in various embodiments, each is within 2 cm of a lateral edge of the bag. For example, in the embodiment of FIG. 1, the first tube 170 is disposed adjacent first lateral edge 134, and the second tube 180 is disposed adjacent second lateral edge 136, which is on an opposite side of the bag from the first lateral edge 134. The tubes can be, for example, substantially parallel to one another, e.g., within 20 degrees or even within 10 degrees of one another. In certain desirable embodiments, the first tube and the second tube extend along the direction of a major axis of the bag. For example, in the embodiment of FIG. 1, the first tube 170 and the second tube 180 extend along the direction of the major axis 132 of the bag. Of course, the tubes can in other embodiments be arranged in any suitable manner. For example, in various embodiments, the first tube and second tubes are curved, and extend along the lateral edges of a rounded bag.

Of course, in other embodiments, tubes are not included. In various embodiments, a filter can be disposed at one or more of the first port and the second port, configured to filter media exiting the bag therethrough. The filter can be configured to allow cells to pass but to prevent microcarriers from passing outside of the bag.

The first, second and (if present) third ports can be located at a variety of positions along the bag. For example, in various embodiments, the first and second ports are formed at edges of the bag, e.g., edges extending orthogonally to the direction of the extension of tubes into the bag. This can be in the same edge as shown in the bag of FIG. 1 or in opposing edges. In embodiments that include tubes as shown in FIG. 1, this allows the relative positions of the first and second ports to facilitate extension of the first liquid-permeable tube and the second liquid-permeable tube along a common direction (e.g., along a major axis of the bag). The third port can be formed, for example, along the same edge as one or both of the first and second port; in the embodiment of FIG. 1, ports 140, 150, and 160 are along a common edge. But the third port can alternatively be along an edge that is orthogonal to the edge(s) in which the first and second ports or formed, or, alternatively along another surface of the bag, e.g., along a bottom surface to allow for gravity-assisted removal of cells. The positions of the ports are not particularly limited, provided the relative positions of the first and second ports (and the first and second tubes coupled thereto) facilitate perfusion through a substantial volume of the bag. The person of ordinary skill in the art will, based on the present disclosure, arrange the first port and the second port, and any structures (such as a first tube and a second tube), e.g., to minimize dead spots in perfusion flow within the bag, and/or to ensure that perfusing flow does not disturb cell growth. When present, the third port can be positioned e.g., to facilitate media sampling and introduction of cells and/or microcarriers into the interior volume of the bag.

To form bags as described herein, edges of the one or more polymer films can be bonded (e.g., to other edges of the same polymer film, or to one another in the case of more than one polymer film) by any desirable method, such as RF welding, thermal impulse welding, ultrasonic welding, hot bar welding, chemical bonding, adhesive bonding, thermal fusion bonding, solvent welding, laser welding, corona discharge, radiation, surface treatment, extreme heat, belt, or melt lamination, etching, plasma treatment, extrusion, wetting, adhesives, or combinations thereof. In certain desirable embodiments, the one or more films are bonded by thermal, laser, or hot bar welding.

When present, the first tube and the second tube can be attached to respective first and second ports formed in one or more edges of the bag by a collar sealing process (e.g., by RF welding, ultrasonic welding, thermal impulse welding, hot bar welding, chemical bonding, adhesive bonding, thermal fusion bonding, solvent welding, laser welding, corona discharge, radiation, extreme heat or melt lamination, etching, plasma treatment, wetting, adhesives, or combinations thereof). This can be done at the time of formation of the bag. The first tube can be sealed (e.g., by collar sealing) such that its outer filter layer can prevent cells from being transmitted from the interior compartment through the tube and out the first port. Thus, the first and/or second tubes can extend from outside the bag into inside the bag (with porous parts thereof desirably extending substantially only in the bag). Of course, in other embodiments, the first and/or second tubes can be terminated at the bag, and be connected through to a fluid system through other tubing; such other tubing can be made from the same materials as described herein for the first and second fluid-permeable tubes.

As noted above, in various embodiments the first tube (e.g., tube 170 of FIG. 1) comprises a first inner support structure defining a central lumen of the tube and a first outer filter layer surrounding the first inner support structure. As described in detail herein, the first inner support structure can take a variety of forms, e.g., a perforated or otherwise porous tube, a frame, or a spiral-wound filament. The support structure can be round in cross-section as shown in the Figures herein, or can have other cross-sectional shapes, e.g., polygonal. In certain desirable embodiments as otherwise described herein, the materials of the support structure and outer filter layer are hydrophilic, self-wetting, and/or of sufficient porosity such that liquid flow through the tube can be initiated or maintained with minimal pressure.

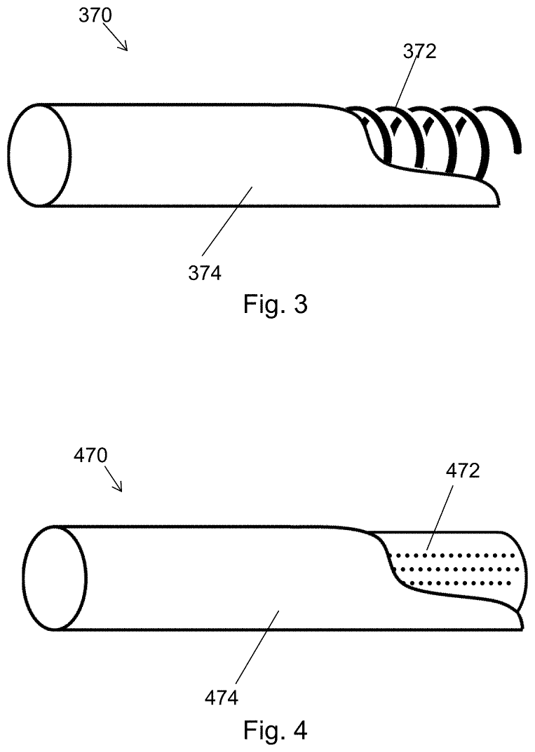

An embodiment of such a fluid-permeable tube is shown in cutaway view in in FIG. 3. Tube 300 of FIG. 3 includes a spiral-wound filament 372 and an outer filter layer 374 surrounding the filament. Outer filter layer 374 is attached to spiral-wound filament 372 at least at the port (e.g., port 140 of FIG. 1) but can be attached to spiral-wound filament 372 at one or more additional points along the filament. The outer filter layer can relatively loosely surround the spiral-wound filament (e.g., as a loose bag over filament 372), or can be disposed more tightly against the spiral-wound filament (e.g., as a close-fitting sleeve over filament 372).

Accordingly, in various embodiments as otherwise described herein, the first inner support structure comprises a spiral-wound filament. The filament can be relatively rigid or relatively flexible and can be wound relatively tightly or relatively loosely, provided, of course, that the filament defines a central lumen of the tube and can support the first outer filter layer. In certain desirable embodiments as otherwise described herein, the filament comprises (e.g., is) a polymer having a total organic carbon in water of less than 0.1 mg/cm². In various embodiments as otherwise described herein, the filament comprises (e.g., is formed of) a fluoropolymer, polymethylpentene, or a combination thereof (e.g., having a total organic carbon in water of less than 0.1 mg/cm²).

In various embodiments as otherwise described herein, the spacing of the spiral-wound filament (i.e., along an axis of the central lumen) is about equivalent to or even larger than the average size of the plurality of microcarriers. For example, in various embodiments as otherwise described herein, the spacing of the spiral-wound filament is at least 500 μm, or within the range of 500 μm to 10 mm, or 500 μm to 7.5 mm, or 500 μm to 5 mm. The present inventors have determined that a tube having a spacing of larger than 100 μm can advantageously facilitate culture media perfusion at a desirable flow rate, without appreciably disturbing anchored cells and/or diminishing the number of contained microcarriers.

In other embodiments as otherwise described herein, a support structure can be provided as a frame, e.g., formed from filament-shaped material, but arranged differently than in a spiral. The filament and spacing of filaments can be as described above for the spiral-wound filament.

In other embodiments as otherwise provided herein, the support structure can be in the form of a porous tube. One such embodiment (e.g., for use as tube 170 of FIG. 1) is shown in cutaway in FIG. 4. Tube 470 of FIG. 4 includes a porous tube 472 and an outer filter layer 474 surrounding the porous tube. The outer filter layer is attached to the porous tube at least at the port (e.g., port 140 of FIG. 1) but can be attached to the porous tube at one or more additional points along the tube. The outer filter layer can relatively loosely surround the porous tube (e.g., as a bag over porous tube 472), or can be disposed more tightly against the porous tube (e.g., as a sleeve over porous tube 472).

Accordingly, in various embodiments as otherwise descried herein, the first inner support structure comprises a first porous tube. The tube can be relatively flexible or relatively rigid, provided, of course, that the tube can support the first outer filter layer. In certain desirable embodiments as otherwise described herein, the porous tube comprises (e.g., is formed of) a polymer having a total organic carbon in water of less than 0.1 mg/cm². In various embodiments as otherwise described herein, the porous tube comprises (e.g., is formed of) an elastomer, fluoropolymer, polymethylpentene, or a combination thereof (e.g., having a total organic carbon in water of less than 0.1 mg/cm²). In certain desirable embodiments as otherwise described herein, the porous tube comprises (e.g., is formed of) silicone or polyvinyl chloride (e.g., having a total organic carbon in water of less than 0.1 mg/cm²). For example, in certain such embodiments, the porous tube comprises a silicone elastomer. In another example, in certain such embodiments, the porous tube comprises fluorinated ethylene propylene. But a variety of fluorinated and non-fluorinated polymers can otherwise be used, as can other suitable materials.

In various embodiments as otherwise described herein, the porous tube has an average pore size within the range of 100 μm to 5,000 μm. For example, in various embodiments as otherwise described herein, the average pore size of the porous tube is 100-2500 μm, or 100-1000 μm, or 250-5000 μm, or 250-2500 μm, or 250-1000 μm, or 500-5000 μm, or 500-2500 μm, or 500-1000 μm. As used herein, average pore size is measured via capillary flow porometry in cases where pores are too small for convenient optical measurement. The person of ordinary skill in the art will recognize that a pore size within the range of 10 μm to 5,000 μm can be about equivalent to or larger than the size of most microcarriers. However, the present inventors have determined that the porous tube can advantageously facilitate culture media perfusion at a desirable flow rate without appreciably disturbing anchored cells and/or diminishing the number of contained microcarriers, especially when used with an outer filter layer.

The porous tube can have a porosity within a broad range; the person of ordinary skill in the art will select a porosity that provides a desired combination of mechanical stability and flow rate. In various embodiments as otherwise described herein, the porous tube has a porosity within the range of 10% to 90%. The person of ordinary skill in the art will appreciate that porosity, a measure of the volume of pores in an article relative to the total volume of the material, can be measured by a number of known porosimetry methods; as used herein, water evaporation is used to measure porosity of the tube in cases where pores are too small for convenient optical measurement. In various embodiments as otherwise described herein, the porosity of the porous tube is within the range of 20% to 90%, or 30% to 90%, or 40% to 90%, or 50% to 90%, or 10% to 80%, or 10% to 70%, or 10% to 60%, or 10% to 50%, or 20% to 80%, or 30% to 70%, or 40% to 60%.

Porosity of the porous tube can be provided by any of a number of art-recognized methods, e.g., molding, thermal perforation, laser drilling, electron beam drilling, electrical discharge machining, mechanical drilling, stamping or cutting.

In certain desirable embodiments as otherwise described herein, a majority of the porosity of the first porous tube is localized on a surface (e.g., a semicylinder surface) opposite the second liquid-permeable tube. Advantageously, the present inventors have determined that orientation of the porosity of the tube away from the second liquid-permeable tube can further minimize disturbance of cultivated cells and/or diminishment of microcarriers effected by culture media perfusion from the second liquid-permeable tube to the first liquid-permeable tube. Accordingly, in various embodiments as otherwise described herein, at least 65%, or at least 75%, or at least 85%, or at least 90% of the porosity of the first porous tube is localized on a surface (e.g., a semicylinder surface) opposite the second liquid-permeable tube.

In certain desirable embodiments as otherwise described herein, the porosity of the first porous tube is distributed relatively uniformly along an axis of the central lumen of the liquid-permeable tube, i.e., to facilitate uniform perfusion along the major axis of the bag.

As described above, the first tube can include a first outer filter layer disposed about the first inner support structure. The first outer filter layer is formed of a porous material that has an average pore size that is selected to help prevent microcarriers from escaping the bag during perfusion. In various embodiments, the average pore size is selected to be smaller than the average size of the microcarriers contained in the bag. In various embodiments as otherwise described herein, the first outer filter layer has an average pore size of less than 200 μm, e.g., less than 150 μm. For example, in various embodiments as otherwise described herein, the average pore size of the first outer filter layer is 10-200 μm, e.g., 20-200 μm, or 30-200 μm, or 75-200 μm, or 10-150 μm, or 20-150 μm, or 30-150 μm, or 50-150 μm, or 75-150 μm. The person of ordinary skill in the art will recognize that a pore size of 200 μm or less can be about equivalent to or smaller than most microcarriers—surprisingly, the present inventors have determined that the combination of the outer filter layer (e.g., having a small functional pore size relative to cultivated cells) and the inner support structure (e.g., having a large functional pore size or spacing relative to cultivated cells) can facilitate perfusion of culture media at a desirable flow rate without appreciably disturbing anchored cells and/or diminishing the number of contained microcarriers. But even when the filter pore size is somewhat larger than the microcarrier size, the filter can be effective in preventing significant loss of microcarriers from the interior volume during perfusion.

In certain desirable embodiments, the first outer filter layer has an average pore size and/or a D99 pore size of less than about 100%, e.g., less than about 75%, or less than about 50%, or less than about 40%, or less than about 30%, or less than about 25% of the average diameter of the microcarriers contained in the bag.

The person of ordinary skill in the art will appreciate that the “functional” pore size of a filter layer depends on both the average size and maximum size of the pores within the layer. Accordingly, in certain desirable embodiments as otherwise described herein, the D99 pore size of the filter layer (i.e., size of the pore that is at the 99^{th} percentile in size) differs from the average pore size of the filter layer by at most 100%, e.g., at most 50%, at most 30%, or at most 10%. In certain desirable embodiments as otherwise described herein, the D99 pore size of the filter layer is less than 500 μm, e.g., less than 250 μm, or less than 200 μm, or less than 150 μm.

However, as the person of ordinary skill in the art will appreciate, a small average pore size of the first outer filter layer can slow down flow through the bag. Accordingly, the person of ordinary skill in the art can select a pore size that is small enough to provide filtration for the desired microcarrier but large enough to provide a desired flow rate through the bag. For example, in certain desirable embodiments as otherwise described therein, the microcarriers contained in the bag have an average diameter of 100-400 μm, and the average pore size of the first outer filter layer is 10-200 μm (e.g., and smaller than the average diameter of the microcarriers).

In certain desirable embodiments as otherwise described herein, the first outer filter layer comprises (e.g., is formed of) a polymer having a total organic carbon in water of less than 0.1 mg/cm². In various embodiments as otherwise described herein, the first outer filter layer comprises one or more of polytetrafluoroethylene (PTFE), polypropylene, polyether ether ketone (PEEK), polyester, nylon, and fluorinated ethylene-propylene. In various embodiments as otherwise described herein, the first outer filter layer comprises stainless steel. In certain such embodiments, the first outer filter layer comprises polymer-coated stainless steel (e.g., polytetrafluoroethylene-coated stainless steel).

However, in other embodiments of the bags as otherwise described herein, the first tube does not include a filter layer separate from a support structure, but instead is a tube that has an average pore size as described herein for the first outer filter layer. This can be made, for example, by forming porous film material into a tube (e.g., by welding), or by providing a rigid tubular material with the desired pore size. In various embodiments as otherwise described herein, the first tube has an average pore size of less than 200 μm, e.g., less than 150 μm. For example, in various embodiments as otherwise described herein, the average pore size of the first tube is 10-200 μm, e.g., 20-200 μm, or 30-200 μm, or 75-200 μm, or 10-150 μm, or 20-150 μm, or 30-150 μm, or 50-150 μm, or 75-150 μm. In certain desirable embodiments, the first tube has an average pore size and/or a D99 pore size of less than about 100%, e.g., less than about 75%, or less than about 50%, or less than about 40%, or less than about 30%, or less than about 25% of the average diameter of the microcarriers contained in the bag, e.g., as described above with respect to the first outer filter layer. In certain desirable embodiments as otherwise described herein, the D99 pore size of the first tube (i.e., size of the pore that is at the 99th percentile in size) differs from the average pore size of the tube by at most 50%, e.g., at most 30%, or at most 10%. In certain desirable embodiments as otherwise described herein, the D99 pore size of the first tube is less than 50 μm, e.g., less than 20 μm. In various embodiments as otherwise described herein, the first tube has a D99 pore size of less than 10 μm. For example, in various embodiments as otherwise described herein, the D99 pore size of the first tube is less than 500 μm, e.g., less than 250 μm, or less than 200 μm, or less than 150 μm. In certain desirable embodiments, the first tube is rigid, i.e., sufficiently rigid to maintain its cross-sectional shape without pressure of a fluid flowing therethrough.

As the second liquid-permeable tube (e.g., tube 180 of FIG. 1) is generally used to input media into the interior compartment of the bag, in many embodiments it need not have a filter layer. Thus, in various embodiments, the second liquid-permeable tube can be as described above for the porous tube support structure of the first liquid-permeable tube, without an outer filter layer. The second liquid-permeable tube can take a variety of other tubular structures.

However, in other embodiments as otherwise described herein, the second liquid-permeable tube comprises a second inner support structure defining a central lumen of the tube and a second outer filter layer surrounding the second inner support structure. And in other embodiments, the second liquid-permeable tube is a tube having an average pore size of no more than 200 μm. In such embodiments, the second liquid-permeable tube can be as otherwise described in any embodiment herein for the first liquid-permeable tube. Advantageously, such bags allow for media flow as described above—from the second tube, through the interior volume, and out of the first tube—as well as the reverse. The present inventors have determined that cells can be cleared from the first outer filter layer, but retained within the interior compartment, by reversing the direction of perfusion in such embodiments. Of course, such “bi-directional” bags can also facilitate more convenient set-up and operation by a user. In certain desirable embodiments, the inner support structure and outer filter layer of the second liquid-permeable tube are as described herein with respect to any embodiment the first liquid-permeable tube. Structures as shown in FIGS. 3 and 4 can in various embodiments be used as the second liquid-permeable tube.

For example, in various embodiments as otherwise described herein, the second inner support structure comprises a spiral-wound filament (e.g., a spiral-wound filament described above). In certain such embodiments, the first inner support structure of the first liquid-permeable tube also comprises a spiral-wound filament. Similarly, in certain other embodiments as otherwise described herein, the second inner support structure comprises a frame structure. In certain such embodiments, the first inner support structure of the first liquid-permeable tube also comprises a frame structure

In another example, in various embodiments as otherwise described herein, the second inner support structure comprises a porous tube (e.g., a porous tube as described above). For example, in various embodiments as otherwise described herein, the first inner support structure comprises a first porous tube and the second inner support structure comprises a second porous tube. In certain such embodiments, the second porous tube (e.g., and the first porous tube) has a functional pore size in the range of 100-2500 μm, or 100-1000 μm, or 250-5000 μm, or 250-2500 μm, or 250-1000 μm, or 500-5000 μm, or 500-2500 μm, or 500-1000 μm. In certain such embodiments, the second porous tube (e.g., and the first porous tube) has a porosity in the range of 10% to 90%, or 20% to 80%, or 30% to 70%. In certain such embodiments, at least 60%, or at least 75%, or at least 90% of the porosity of the second porous tube is localized on a surface (e.g., a semicylinder surface) opposite the first liquid-permeable tube (e.g., and at least 60%, or at least 75%, or at least 90% of the porosity of the first porous tube is localized on a surface (e.g., a semicylinder surface) opposite the second liquid-permeable tube). In various embodiments, the second porous tube (e.g., and the first porous tube) comprises a silicone elastomer. In various embodiments, the second outer filter layer (e.g., and the first outer filter layer) has an average pore size of less than 200 μm, e.g., less than 150 μm. In various embodiments as otherwise described herein, the average pore size of the second outer filter layer is 10-200 μm, e.g., 20-200 μm, or 30-200 μm, or 75-200 μm, or 10-150 μm, or 20-150 μm, or 30-150 μm, or 50-150 μm, or 75-150 μm. In various embodiments, the second outer filter layer (e.g., and the first outer filter layer) comprises one or more of stainless steel, polytetrafluoroethylene (PTFE), polypropylene, polyether ether ketone (PEEK), polyester, nylon, and fluorinated ethylene-propylene.

And in other embodiments, the second liquid-permeable tube is a tube that does not include a second support structure and a second outer filter layer, but instead is in the form of a tube having a pore size as described herein for the second outer filter layer. This can be made, for example, by forming porous film material into a tube (e.g., by welding), or by providing a rigid tubular material with the desired pore size. In various embodiments as otherwise described herein, the second tube has an average pore size of less than 200 μm, e.g., less than 150 μm. For example, in various embodiments as otherwise described herein, the average pore size of the second tube is 10-200 μm, e.g., 20-200 μm, or 30-200 μm, or 75-200 μm, or 10-150 μm, or 20-150 μm, or 30-150 μm, or 50-150 μm, or 75-150 μm. In certain desirable embodiments, the second tube has an average pore size and/or a D99 pore size of less than about 100%, e.g., less than about 75%, or less than about 50%, or less than about 40%, or less than about 30%, or less than about 25% of the average diameter of the microcarriers contained within the bag, e.g., as described above with respect to the first outer filter layer. In certain desirable embodiments as otherwise described herein, the D99 pore size of the second tube (i.e., size of the pore that is at the 99^{th} percentile in size) differs from the average pore size of the second tube by at most 50%, e.g., at most 30%, or at most 10%, In certain desirable embodiments, the second tube is rigid, i.e., sufficiently rigid to maintain its cross-sectional shape without pressure of a fluid flowing therethrough.

In cases where the second liquid-permeable tube is covered by a second filter layer or has a small pore size as described above, then it can be desirable for the third port to be present to simplify the introduction of cells into and/or the removal of cells from the interior volume of the bag.

As noted above, the present inventors contemplate that filtration at the first and/or second port can be provided by other than the tubular structures provided herein. For example, a filter can be operatively coupled to the first port, and/or a filter can be operatively coupled to the second port. In other examples, a filter can be inset into a connector that acts to connect a port to other parts of a cell culture system. These filters can generally have the same properties as those described above. For example, In various embodiments, the average pore size is selected to be smaller than the average size of the microcarriers contained in the bag. In various embodiments as otherwise described herein, the filter has an average pore size of less than 200 μm, e.g., less than 150 μm. For example, in various embodiments as otherwise described herein, the average pore size of the filter is 10-200 μm, e.g., 20-200 μm, or 30-200 μm, or 75-200 μm, or 10-150 μm, or 20-150 μm, or 30-150 μm, or 50-150 μm, or 75-150 μm The person of ordinary skill in the art will recognize that a pore size of 200 μm or less can be about equivalent to or smaller than most microcarriers—but even when the filter pore size is somewhat larger than the microcarrier size, the filter can be effective in preventing significant loss of microcarriers from the interior volume during perfusion. In certain desirable embodiments, the filter has an average pore size and/or a D99 pore size of less than about 100%, e.g., less than about 75%, or less than about 50%, or less than about 40%, or less than about 30%, or less than about 25% of the average diameter of the microcarriers contained in the bag. The person of ordinary skill in the art will appreciate that the “functional” pore size of a filter depends on both the average size and maximum size of the pores within the layer. Accordingly, in certain desirable embodiments as otherwise described herein, the D99 pore size of the filter (i.e., size of the pore that is at the 99^{th} percentile in size) differs from the average pore size of the filter by at most 100%, e.g., at most 50%, at most 30%, or at most 10%. In certain desirable embodiments as otherwise described herein, the D99 pore size of the filter is less than 500 μm, e.g., less than 250 μm, or less than 200 μm, or less than 150 μm. However, as the person of ordinary skill in the art will appreciate, a small average pore size of the filter can slow down flow through the bag. Accordingly, the person of ordinary skill in the art can select a pore size that is small enough to provide filtration for the desired microcarrier but large enough to provide a desired flow rate through the bag. For example, in various desirable embodiments as otherwise described therein, the microcarriers contained in the bag have an average diameter of 100-400 μm, and the average pore size of the filter is 50-200 μm (e.g., and smaller than the average diameter of the microcarriers).

As noted above, the present inventors have determined that the bags described herein are useful for perfusing media through a microcarrier cell culture. Accordingly, another aspect of the disclosure is a method for cultivating cells that includes providing a bag as otherwise described herein (e.g., bag 100 of FIG. 1), including media and cells (e.g., as otherwise described herein) in the interior compartment (e.g., interior compartment 130), and flowing media through the interior compartment by adding media through the second port (e.g., port 150) (and through the second liquid-permeable tube (e.g., tube 180), and removing media the first port (e.g., port 140) (if present, via the first liquid-permeable tube (e.g., tube 170)). In various such embodiments, the bag including media and cells in the interior compartment can be provided by adding media and cells to the bag through a third port formed in an exterior surface of the bag and in fluid communication with the interior compartment of the bag (e.g., port 160). However, in other embodiments, especially when no third port is present, the first or second port can be used to admit media and cells to the bag. A variety of flow rates can be used, for example, in the range of 0.5-20 mL/min; the flow rate is desirably low enough to allow microcarriers to remain suspended in the culture media. Of course, over the course of normal operation, flow rates can vary, or can even be discontinuous (e.g., incidentally, or based on cell activity). Accordingly, in another example, the flow rate can in the range of 0.2-2 bag volume replacements per day. The bag can be maintained at a desired incubation temperature during the perfusion, and can be rocked or otherwise agitated.

In various embodiments as otherwise described herein, the method includes, after an incubation period (e.g., sufficient to increase the number of microcarrier-adhered cells to a desired first level), adding an additional plurality of microcarriers to the bag (e.g., to increase the total area of adhesion substrate available for cultivated cells). In various embodiments, the additional plurality of microcarriers (e.g., comprising microcarriers as otherwise described herein) through a third port at in an exterior surface of the bag and in fluid communication with the interior compartment of the bag (e.g., port 160).

In various embodiments as otherwise described herein, the method further comprises separating cultivated cells from the microcarriers (e.g., alter the number of microcarrier-adhered cells increases to a desired, final level), and then removing the cultivated cells from the interior compartment of the bag. As the person of ordinary skill in the art will appreciate, methods for separating adherent cells from microcarriers are known in the art. For example, in various embodiments as otherwise described herein, separating cultivated cells from the microcarriers comprises adding trypsin to the feed media (e.g., flowed through the second port)

The present inventors note that, advantageously, cultivated cells can be removed from the interior compartment of the bag through the first port (e.g., port 140) (if present, via the first liquid-permeable tube (e.g., tube 170)), while the microcarriers are retained in the interior compartment for example, by a filter operatively coupled to the first port, be it a first outer filter layer disposed about a first tube, a first porous tube, or some other filter.

However, the present inventors note that a filter operatively coupled with respect to the first port or the second port is not required in the systems and methods of the disclosure. In some embodiments, the flow can be controlled to avoid pushing a substantial portion of the microcarriers out of the bag. However in other embodiments, a porous membrane can be used to separate volume(s) of the interior compartment that communicates the first and second ports from a volume of the interior compartment in which the microcarriers are disposed. Even when the pore size of the membrane is somewhat larger than the size of the microcarriers, the membrane can allow for exchange of media between the compartments without allowing the flow between the first and second ports to disturb and carry away the microcarriers.

Accordingly, in another example, a bag as described herein can include a perfusive membrane. In various embodiments as otherwise described herein, the bag comprises a first porous membrane separating a first portion of the interior compartment from a second portion of the interior compartment, the second portion containing the plurality of microcarriers, and a third port formed in an exterior surface of the bag. In such embodiments, the first port and the second port are each in fluid communication with the first portion of the interior compartment, and the third port is in fluid communication with the second portion of the interior compartment. The present inventors note that, desirably, feed media can be perfused through the first portion of the interior compartment of the bag without appreciably disturbing anchored cells and/or diminishing the number of microcarriers contained in the second portion of the interior compartment. The present inventors note that, desirably, feed media can be perfused through the interior compartment of the bag without appreciably disturbing anchored cells and/or diminishing the number of contained microcarriers by flowing media through the interior volume between the first and second ports. The porous membrane dividing the interior compartment can prevent microcarriers from escaping the second portion of the interior compartment. Anchorage-dependent cells can thus be grown and concentrated in the second portion without feed media supply limitations and/or waste removal limitations. The third port can be used for media sampling and introduction of cells and/or microcarriers into the second portion of the interior compartment. The present inventors moreover note that, advantageously, following a cell culture process, cultivated cells can be detached from the microcarriers and removed from the bag through the porous membrane (i.e., which retains the microcarriers in the interior compartment). In another embodiment, the third port can have a filter operatively coupled thereto as described above with respect to the first and second ports, which can allow cells to be harvested without removal of the microcarriers from the bag.

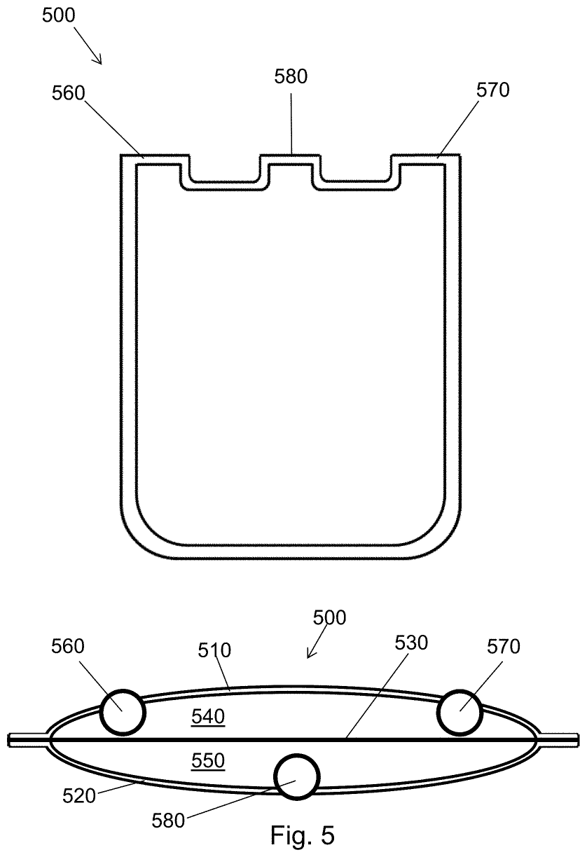

An embodiment of such a bag is shown in schematic top-down view (top) and cross-sectional view (bottom) in FIG. 5. Bag 500 of FIG. 5 includes a first polymer film 510 and a second polymer film 520, bonded together at edges thereof to form edges around an interior compartment, the interior compartment separated by a porous membrane 530 into a first portion 540 and a second portion 550. Bag 500 further includes first port 560 and second port 570, each in fluid communication with the first portion 540 of the interior compartment, and a third port 580 in fluid communication with the second portion 550 of the interior compartment.

The bag is desirably configured to allow perfusion of media through a substantial portion of the interior volume by flowing between the first port and the second port. The person of ordinary skill in the art will, based on the present disclosure, arrange the first port and the second port, e.g., to minimize dead spots in perfusion flow within the bag, and/or to ensure that perfusing flow does not disturb cell growth. The third port can be positioned, e.g., to facilitate media sampling and introduction of cells and/or microcarriers into the second portion of the bag.

In another example (not shown), the bag comprises a first liquid-permeable tube (e.g., as otherwise described herein) extending into and in fluid communication with the first portion, the first liquid-permeable tube being operatively coupled to the first port, and a second liquid-permeable tube (e.g., as otherwise described herein) extending into and in fluid communication with the first portion, the second liquid-permeable tube being operatively coupled to the second port. In certain such embodiments, the tubes can be, for example, substantially parallel to one another and disposed adjacent opposed edges of the bag (e.g., increasing the distribution of flowing media over the porous membrane).

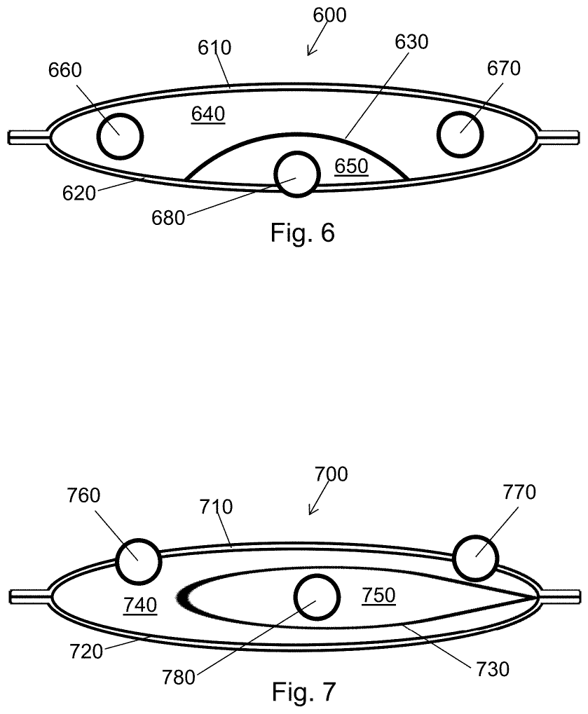

In the embodiment of FIG. 5, the interior compartment is divided into a first portion 540 bounded by the first polymer film 510 and the porous membrane 520 and a second portion 550 bounded by the second polymer film 520 and the porous membrane 530. Of course, the interior compartment need not be so evenly divided. For example, in the embodiment of FIG. 6, bag 600 includes a first polymer film 610 and a second polymer film 620, bonded together at edges thereof to form edges around an interior compartment, and a porous membrane 630 having edges bonded to the second polymer film 620, separating the interior compartment into a first portion 640 and a second portion 650. In another example, in the embodiment of FIG. 7, bag includes a first polymer film 710 and a second polymer film 720, bonded together at edges thereof to form edges around an interior compartment, and a porous membrane 730, folded over and bonded at edges thereof to separate the interior compartment into a first portion 740 and second portion 750. In such embodiments, the person of ordinary skill in the art can position the first port, second port, and third ports described herein, e.g., to minimize dead spots in perfusion flow within the bag, to ensure that perfusing flow does not disturb cell growth, to facilitate introduction of cells and/or microcarriers to the second portion of the bag, and/or to provide for a desired position of fluid sampling or monitoring in the bag.

In yet another example, in various embodiments as otherwise described herein, the bag comprises a first porous membrane separating a first portion of the interior compartment from a central portion of the interior compartment, a second porous membrane separating a second portion of the interior compartment form the central portion of the interior compartment, the central portion containing the microcarriers, and a third port formed in an exterior surface of the bag. In such embodiments, the first port is in fluid communication with the first portion of the interior compartment, the second port is in fluid communication with the second portion of the interior compartment, and the third port is in fluid communication with the central portion of the interior compartment. The present inventors note that, desirably, feed media can be perfused through the central portion of the interior compartment of the bag without appreciably disturbing anchored cells and/or diminishing the number of microcarriers contained in the central portion of the interior compartment. The present inventors note that, desirably, feed media can be perfused through the interior compartment of the bag without appreciably disturbing anchored cells and/or diminishing the number of contained microcarriers by flowing media from the second tube, through the interior volume, and out of the first tube. The porous membrane dividing the interior compartment can prevent microcarriers from escaping the central portion of the interior compartment. Anchorage-dependent cells can thus be grown and concentrated in the central portion without feed media supply limitations and/or waste removal limitations. The third port can be used for media sampling and introduction of cells and/or microcarriers into the central portion of the interior compartment. The present inventors moreover note that, advantageously, following a cell culture process, cultivated cells can be detached from the microcarriers and removed from the bag through the porous membrane (i.e., which retains the microcarriers in the central compartment).

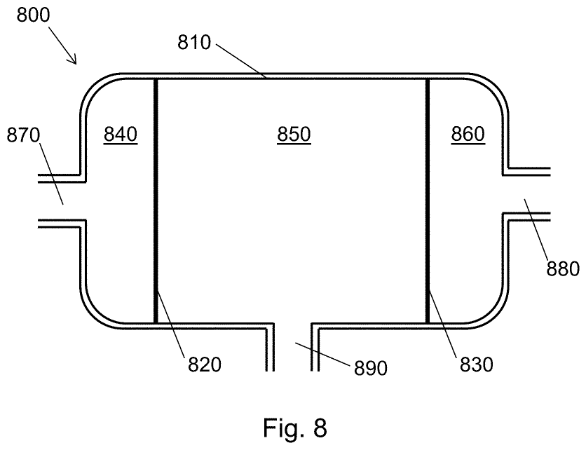

An embodiment of such a bag is shown in schematic top-down view in FIG. 8. Bag 800 of FIG. 8 includes a first polymer film 810 and a second polymer film (not shown), bonded together at edges thereof to form edges around an interior compartment, the interior compartment separated by a first porous membrane 820 into a first lateral portion 840 and a central portion 850, and separated by a second porous membrane 830 into a second lateral portion 860 and the central portion 850. Bag 800 further includes a first port 870 in fluid communication with the first lateral portion 840 of the interior compartment, a second port 880 in fluid communication with the second lateral portion 860, and a third port 890 in fluid communication with the central portion 850 of the interior compartment.

The bag is desirably configured to allow perfusion of media through a substantial portion of the interior volume by flowing from the second port to the first port. The person of ordinary skill in the art will, based on the present disclosure, arrange the first port and the second port, e.g., to minimize dead spots in perfusion flow within the bag, and/or to ensure that perfusing flow does not disturb cell growth. The third port can be positioned, e.g., to media sampling and introduction of cells and/or microcarriers into the central portion of the bag.

To form bags including one or more membranes dividing the interior compartment, edges of one or more polymer films and one or more porous membranes can be bonded by any desirable method, such as RF welding, thermal impulse welding, ultrasonic welding, hot bar welding, chemical bonding, adhesive bonding, thermal fusion bonding, solvent welding, laser welding, corona discharge, radiation, surface treatment, extreme heat, belt, or melt lamination, etching, plasma treatment, extrusion, wetting, adhesives, or combinations thereof. In certain desirable embodiments, the polymer films are bonded together by thermal, laser, or hot bar welding.

In certain desirable embodiments as otherwise described herein, the one or more porous membranes comprise (e.g., are formed of) a polymer having a total organic carbon in water of less than 0.1 mg/cm². In certain desirable embodiments as otherwise described herein, the one or more porous membranes each individually comprise substantially fluoropolymer, e.g., at east 80 wt. %, or at east 85 wt. %, or at east 90 wt. %, or at least 95 wt. %, or at least 97.5 wt. %, or at least 98 wt. %, or at least 99 wt. % fluoropolymer (e.g., as described above with respect to the inner layer of the one or more polymer films)

In various embodiments as otherwise described herein, the one or more porous membranes each individually comprise one or more fluoropolymers selected from ethylene-propylene (FEP), polytetrafluoroethylene (PTFE), polyvinylidenefluoride (PVDF), tetrafluoroethylene-perfluoro(propyl vinyl ether) (PFA), polyvinylidene difluoride (PVF), polychlorotrifluoroethlylene (PCTFE), tetrafluoroethylene/hexafluoropropylene/ethylene copolymers (HTE), chlorotrifluoroethylene/vinylidenefluoride copolymers, chlorotrifluoroethylene/hexafluoropropylene copolymers, ethylene/chlorotrifluoroethylene copolymers (ECTFE), ethylene/trifluoroethylene copolymers, ethylene/tetrafluoroethylene copolymers (ETFE), tetrafluoroethylene/propylene copolymers (TFE/P), tetrafluoroethylene/hexafluoropropylene copolymers (FEP/HFP), hexafluoropropylene/tetrafluoroethylene/vinylidene copolymers (THV), and perfluoro(1-butenyl vinyl ether) homocyclopolymers having functionalized polymer-end groups. In certain desirable embodiments, the one or more porous membranes each individually comprise at least 80 wt. % (e.g., at least 90 wt. %, or at least 95 wt. %) fluorinated ethylene-propylene.

In various embodiments as otherwise described herein, the one or more porous membranes each individually comprise one or more of polytetrafluoroethylene (PTFE), polypropylene, polyether ether ketone (PEEK), polyester, nylon, and fluorinated ethylene-propylene. In various embodiments as otherwise described herein, one or more porous membranes comprises stainless steel. In various embodiments as otherwise described herein, one or more porous membranes comprise a fluoropolymer-coated substrate. For example, in certain such embodiments, one or more porous membranes comprise fluoropolymer-coated stainless steel (e.g., polytetrafluoroethylene-coated stainless steel).

In various embodiments as otherwise described herein, the one or more porous membranes (e.g., comprising substantially fluoropolymer) each individually have a thickness of 0.001-0.7 mm, e.g., 0.001-0.4 mm, or 0.001-0.1 mm, or 0.005-0.7 mm, or 0.005-0.4 mm, or 0.005-0.1 mm, or 0,01-7 mm, or 0.01-0.4 mm, or 001-0.1 mm. In certain desirable embodiments as otherwise described herein the one or more porous membranes each individually comprise at least 95 wt. % fluorinated ethylene-propylene, and have a thickness of 0.01-0.1 mm.

In various embodiments as otherwise described herein, the one or more porous membranes each have an average pore size that is selected to help prevent microcarriers from escaping the second portion or central portion during perfusion. In various embodiments as otherwise described herein, the average pore size the one or more porous membranes is less than less than 200 μm, e.g., less than 150 μm. For example, in various embodiments as otherwise described herein, the average pore size of the one or more porous membranes is 10-200 μm, e.g., 20-200 μm, or 30-200 μm, or 75-200 μm, or 10-150 μm, or 20-150 μm, or 30-150 μm, or 50-150 μm, or 75-150 μm.

In certain desirable embodiments, the one or more porous membranes has an average pore size and/or a D99 pore size of less than about 100%, e.g., less than about 75%, or less than about 50%, or less than about 40%, or less than about 30%, or less than about 25% of the average diameter of the microcarriers contained in the bag.

The one or more porous membranes can have a porosity within a broad range; the person of ordinary skill in the art will select a porosity that provides a desired combination of mechanical stability and flow rate. In various embodiments as otherwise described herein, the one or more porous membranes each individually have a porosity within the range of 10% to 90%. In various embodiments as otherwise described herein, the porosity of the porous tube is within the range of 20% to 90%, or 30% to 90%, or 40% to 90%, or 50% to 90%, or 10% to 80%, or 10% to 70%, or 10% to 60%, or 10% to 50%, or 20% to 80%, or 30% to 70%, or 40% to 60%.

Porosity of the porous membrane can be provided by any of a number of art-recognized methods, e.g., molding, thermal perforation, laser drilling, electron beam drilling, electrical discharge machining, mechanical drilling, stamping or cutting.

The person of ordinary skill in the art will appreciate that the “functional” pore size of a porous membrane depends on both the average size and maximum size of the pores within the membrane. Accordingly, in certain desirable embodiments as otherwise described herein, the D99 pore size of the one or more porous membranes (i.e., size of the pore that is at the 99^{th} percentile in size) differs from the average pore size of the one or more porous membranes by at most 100%, e.g., at most 50%, at most 30%, or at most 10%. In certain desirable embodiments as otherwise described herein, the D99 pore size of the one or more porous membranes is less than 500 μm, e.g., less than 250 μm, or less than 200 μm, or less than 150 μm.

However, as the person of ordinary skill in the art will appreciate, a small average pore size of the one or more porous membranes can slow down flow through the bag and/or slow down exchange between the portion of the interior compartment through which media is perfused and the portion of the interior compartment in which the microcarriers are disposed. Accordingly, the person of ordinary skill in the art can select a pore size that is small enough to provide filtration for the desired in microcarrier but large enough to provide a desired flow rate through the bag. For example, in certain desirable embodiments as otherwise described therein, the microcarriers contained in the bag have an average diameter of 100-400 μm, and the average pore size of the one or more porous membranes is 50-200 μm (e.g., and smaller than the average diameter of the microcarriers). But in other embodiments (e.g., embodiments analogous to embodiments of FIGS. 5-7) the average pore diameter of the one or more porous membranes can be somewhat larger than the average diameter of the microcarriers. In such cases the membrane can nonetheless prevent a significant proportion of microcarriers from escape.

As noted above, the present inventors have determined that the bags described herein are useful for perfusing media through a cell culture. Accordingly, another aspect of the disclosure is a method for cultivating cells that includes providing anchorage-dependent cells and media together with the microcarriers in the interior compartment of a bag as otherwise described herein (e.g., bag 500 of FIG. 5 or bag 800 of FIG. 8) and flowing media through the interior compartment between the first port and the second port. In cases where a filter is operatively coupled to the first port, the flow can be, e.g., from the second port to the first port. In cases where one or more membranes divide the interior compartment into various compartments, a third port can be used for admitting the microcarriers to the bag; cells and media can be added through the third port as well.

A variety of flow rates can be used, for example, in the range of 0.5-20 mL/min; the flow rate is desirably low enough to allow microcarriers to remain suspended in the culture media. Of course, over the course of normal operation, flow rates can vary, or can even be discontinuous (e.g., incidentally, or based on cell activity). Accordingly, in another example, the flow rate can in the range of 0.2-2 bag volume replacements per day. The bag can be maintained at a desired incubation temperature during the perfusion, and can be rocked or otherwise agitated.

In various embodiments as otherwise described herein, the method includes, after an incubation period (e.g., sufficient to increase the number of microcarrier-adhered cells to a desired first level), adding an additional plurality of microcarriers to the bag (e.g., to increase the total area of adhesion substrate available for cultivated cells). In various embodiments, the additional plurality of microcarriers (e.g., comprising microcarriers as otherwise described herein) through a third port, or, in embodiments where a filter is operatively coupled to the first port, through the second port.

In various embodiments as otherwise described herein, the method further comprises separating cultivated cells from the microcarriers (e.g., after the number of microcarrier-adhered cells increases to a desired, final level), and then removing the cultivated cells from the interior compartment of the bag. As the person of ordinary skill in the art will appreciate, methods for separating adherent cells from microcarriers are known in the art. For example, in various embodiments as otherwise described herein, separating cultivated cells from the microcarriers comprises adding trypsin to the feed media (e.g., flowed through the second port).

The present inventors note that, advantageously, cultivated cells can be removed from the interior compartment of the bag, using a filter or porous membrane to prevent significant quantities of microcarriers from exiting the bag. That is, the microcarriers are retained in the interior compartment by the porous membrane or filter.

### EXAMPLES

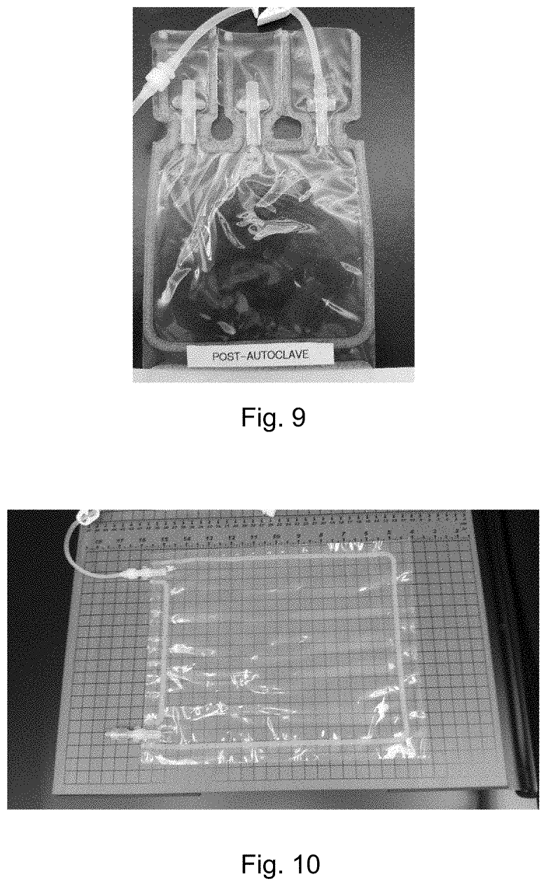

In an example, an oxygen- and carbon dioxide-permeable bag A was formed from polymer films comprising an inner fluorinated ethylene-propylene layer and an outer silicone rubber layer. Bag A and a comparative, commercially available fluoropolymer bag C were sterilized in an autoclave under similar, standard conditions (121° C. for 30 minutes, followed by 60 minutes of drying time). As demonstrated in FIG. 9, Bag C shrunk significantly (2-5% in the film extrusion “machine direction,” the vertical direction as pictured; 1-3% in the film extrusion “transverse direction,” the horizontal direction as pictured) causing the inner surface of the bag to become visibly wrinkled. In contrast, Bag A remained desirably smooth, as shown in FIG. 10.

Polystyrene microcarriers, media, and adherent cells were added to the sterilized bags, which were then incubated on a rocking device. After an initial incubation period, the microcarrier distribution was more evenly distributed throughout the volume of bag A than bag C (which showed significant “pooling” of microcarriers between wrinkles of the inner surface of the bag). And the total yield of cultivated cells (per culture volume) from bag A (i.e., after separation from the microcarriers) was significantly higher than that of bag C. Without being bound by theory, the present inventors note that microcarriers (and accordingly, adhered cells) in bag A could be subjected to significantly less shear force and collision force than those in bag C, where microcarriers can collide with wrinkles in the surface of bag C, and can collide with other “pooled” microcarriers at significantly higher rates than those in the even distribution of bag A.

Additional aspects of the disclosure are provided by the following non-limiting enumerated embodiments, which can be combined in any number and in any combination that is not technically or logically inconsistent.

### Embodiment 1

An oxygen-permeable bag comprising
one or more polymer films defining a boundary of an interior compartment of the bag,
   each of the one or more polymer films comprising
      an inner layer adjacent the interior compartment of the bag, the inner layer comprising a fluoropolymer; and
      adhered to the inner layer, an outer layer comprising a polymer;
a first port formed in an exterior surface of the bag and in fluid communication with the interior compartment;
a second port formed in an exterior surface of the bag and in fluid communication with the interior compartment; and
contained in the interior compartment, a plurality of microcarriers.

### Embodiment 2

The bag of Embodiment 1, wherein the oxygen-permeable bag is an oxygen-permeable bag comprising
one or more polymer films having edges bonded to form an interior compartment of the bag, each of the one or more polymer films comprising an inner layer adjacent the interior compartment of the bag, the inner layer comprising a fluoropolymer, and adhered to the inner layer, an outer layer comprising a polymer;
a first port formed in an exterior surface of the bag and in fluid communication with the interior compartment;
a second port formed in an exterior surface of the bag and in fluid communication with the interior compartment; and
contained in the interior compartment, a plurality of microcarriers.

### Embodiment 3

The bag of any of Embodiments 1-2, wherein the inner layer comprises one or more of fluorinated ethylene-propylene (FEP), polytetrafluoroethylene (PTFE), polyvinylidenefluoride (PVDF), tetrafluoroethylene-perfluoro(propyl vinyl ether) (PFA), polyvinylidene difluoride (PVF), polychlorotrifluoroethlylene (PCTFE), a tetrafluoroethylene/hexafluoropropylene/ethylene copolymer (HTE), a chlorotrifluoroethylene/vinylidenefluoride copolymer, a chlorotrifluoroethylene/hexafluoropropylene copolymer, an ethylene/chlorotrifluoroethylene copolymer (ECTFE), an ethylene/trifluoroethylene copolymer, an ethyleneltetrafluoroethylene copolymer (ETFE), a tetrafluoroethylene/propylene copolymer (TFE/P), a tetrafluoroethylene/hexafluoropropylene copolymer (FEP/HFP), a hexafluoropropylene/tetrafluoroethylene/vinylidene copolymer (THV), or a perfluoro(1-butenyl vinyl ether) homocyclopolymer having functionalized polymer-end groups.

### Embodiment 4

The bag of any of Embodiments 1-2, wherein the inner layer comprises fluorinated ethylene-propylene.

### Embodiment 5

The bag of any of Embodiments 1-4, wherein the inner layer has a thickness of 0.001-0.7 mm (e.g., 0.01-0.1 mm).

### Embodiment 6

The bag of any of Embodiments 1-5, wherein the outer layer comprises an elastomer.

### Embodiment 7

The bag of any of Embodiments 1-5, wherein the outer layer comprises one or more of natural polyisoprene rubber (NR), synthetic polyisoprene rubber (IR), polybutadiene rubber (BR), chloroprene rubber (CR), butyl rubber (HR), halogenated butyl rubbers (CIIR, BIIR), styrene-butadiene rubber (SBR), nitrile rubber (NBR) and hydrogenated nitrile rubber (HNBR), ethylene propylene rubber (EPM), ethylene propylene diene rubber (EPDM), epichlorohydrin rubber (ECO), polyacrylic rubber (ACM, ABR), silicone rubber (SI, Q, VMQ), fluorosilicone rubber (FSR, FVMQ), fluoroelastomers (FKM, FEPM), perfluoroelastomers (FFKM), polyether block amides (PEBA), chlorosulfonated polyethylene (CSM), ethylene-vinyl acetate (EVA), cyclic olefin copolymers, polyolefin elastomers, and elastomeric PET.

### Embodiment 8

The bag of any of Embodiments 1-5, wherein the outer layer comprises polymethylpentene polymer (PMP).

### Embodiment 9

The bag of any of Embodiments 1-5, wherein the outer layer comprises silicone rubber (e.g., selected from high consistency rubber (HCR), fluorosilicone rubber (FSR), liquid silicone rubber (LSR), room temperature vulcanized rubber (RTV), thermoplastic silicone rubber (TPE), platinum-cured silicone rubber, and peroxide-cured silicone rubber).

### Embodiment 10

The bag of any of Embodiments 1-9, wherein the outer layer has a thickness of 0.01-5 mm (e.g., 0.05-1 mm).

### Embodiment 11

The bag of any of Embodiments 1-2, wherein
the inner layer has a thickness of 0.001-0.7 mm (e.g., 0.01-0.1 mm) and comprises substantially (e.g., at least 95 wt. %) fluorinated ethylene-propylene; and
the outer layer has a thickness of 0.01-5 mm (e.g., 0.1-0.5 mm) and comprises substantially (e.g., at least 95 wt. %) silicone rubber.

### Embodiment 12

The bag of any of Embodiments 1-11, wherein the inner layer and the outer layer are bonded (e.g., by one or more of chemical bonding, adhesive bonding, thermal fusion bonding, solvent bonding, laser welding, surface treatment, extrusion, co-extrusion, coating, and lamination).

### Embodiment 13

The bag of any of Embodiments 1-12, wherein the microcarriers have an average diameter of 100-400 μm (e.g., 110-300 μm, or 120-275 μm).

### Embodiment 14

The bag of any of Embodiments 1-13, wherein the microcarriers have an average density of 1-1.25 g/mL (e.g., 1-1.15 g/mL, or 1-1.1 g/mL).

### Embodiment 15

The bag of any of Embodiments 1-14, wherein the microcarriers have an average specific surface area of 0.1-10 cm²/mg (e.g., 0.5-7.5 cm²/mg, or 1-7.5 cm²/mg).

### Embodiment 16

The bag of any of Embodiments 1-15, wherein the microcarriers comprise polystyrene, cross-linked dextran, or cellulose.

### Embodiment 17

The bag of any of Embodiments 1-16, wherein the microcarriers comprise one or more extracellular matrix compounds (e.g., selected from collagen I, poly-L-lysine, fibronectin, retronectin, hyaluronic acid, and polydopamine), the extracellular matrix compounds making up at least a portion of a surface of the microcarriers.

### Embodiment 18

The bag of any of Embodiments 1-17, wherein the plurality of microcarriers contained in the interior compartment has a total surface area that is at least 100% (e.g., at least 500%, or at least 1000%) of a total surface area of the interior compartment of the bag.

### Embodiment 19

The bag of any of Embodiments 1-18, comprising
an undivided interior compartment;
a first liquid-permeable tube extending into and in fluid communication with the interior compartment, the first liquid-permeable tube being operatively coupled to the first port; and
a second liquid-permeable tube extending into and in fluid communication with the interior compartment, the second liquid-permeable tube being operatively coupled to the second port;
   wherein the first liquid-permeable tube comprises a first inner support structure defining a central lumen of the tube and a first outer filter layer surrounding the first inner support structure.

### Embodiment 20

The bag of Embodiment 19, comprising a third port formed in an exterior surface of the bag and in fluid communication with the interior compartment of the bag.

### Embodiment 21

The bag of Embodiment 19 or Embodiment 20, wherein the first inner support structure comprises a first porous tube.

### Embodiment 22

The bag of Embodiment 21, wherein the first porous tube has an average pore size of 100-5,000 μm, and a porosity of 10-90%.

### Embodiment 23

The bag of Embodiment 19 or Embodiment 20, wherein the first inner support structure comprises a spiral-wound filament.

### Embodiment 24

The bag of any of Embodiments 19-23, wherein the first outer filter layer comprises one or more of stainless steel, polytetrafluoroethylene (PTFE), polypropylene, polyether ether ketone (PEEK), polyester, nylon, and fluorinated ethylene-propylene.

### Embodiment 25

The bag of any of Embodiments 19-24, wherein the first outer filter layer has an average pore size of 50-200 μm (e.g., 50-150 μm, or 75-150 μm).

### Embodiment 26

The bag of any of Embodiments 19-25, wherein the second liquid-permeable tube comprises a second inner support structure defining a central lumen of the tube and a second outer filter layer surrounding the second inner support structure.

### Embodiment 27

The bag of Embodiment 26, wherein the second inner support structure comprises a spiral-wound filament or a second porous tube.

### Embodiment 28

The bag of Embodiment 26 or Embodiment 27, wherein the second outer filter layer comprises one or more of stainless steel, polytetrafluoroethylene (PTFE), polypropylene, polyether ether ketone (PEEK), polyester, nylon, and fluorinated ethylene-propylene.

### Embodiment 29

The bag of any of Embodiments 28-28, wherein the second outer filter layer has an average pore size of 50-200 μm (e.g., 50-150 μm, or 75-150 μm).

### Embodiment 30

The bag of any of Embodiments 1-18, comprising
an undivided interior compartment;
a first liquid-permeable tube extending into and in fluid communication with the interior compartment of the bag, the first liquid-permeable tube being operatively coupled to the port; and
a second liquid-permeable tube extending into and in fluid communication with the interior compartment of the bag, the second liquid-permeable tube being operatively coupled to the second port;
   wherein the first liquid-permeable tube has an average pore size of 50-200 μm (e.g., 50-150 μm, or 75-150 μm).

### Embodiment 31

The bag of Embodiment 30, wherein the first liquid-permeable tube comprises one or more of stainless steel, polytetrafluoroethylene (PTFE), polypropylene, polyether ether ketone (PEEK), polyester, nylon, and fluorinated ethylene-propylene.

### Embodiment 32

The bag of any of Embodiments 1-18, comprising
a porous membrane separating a first portion of the interior compartment from a second portion of the interior compartment, the second portion containing the plurality of microcarriers; and
a third port formed in an exterior surface of the bag;
   wherein the first port and second port are each in fluid communication with the first portion of the interior compartment, and the third port is in fluid communication with the second portion of the interior compartment.

### Embodiment 33

The bag of Embodiment 32, wherein the porous membrane comprises a fluoropolymer.

### Embodiment 34

The bag of Embodiment 32, wherein the porous membrane comprises one or more of fluorinated ethylene-propylene (FEP), polytetrafluoroethylene (PTFE), polyvinylidenefluoride (PVDF), tetrafluoroethylene-perfluoro(propyl vinyl ether) (PFA), polyvinylidene difluoride (PVF), polychlorotrifluoroethlylene (PCTFE), a tetrafluoroethylene/hexafluoropropylene/ethylene copolymer (HTE), a chlorotrifluoroethylene/vinylidenefluoride copolymer, a chlorotrifluoroethylene/hexafluoropropylene copolymer, an ethylene/chlorotrifluoroethylene copolymer (ECTFE), an ethylene/trifluoroethylene copolymer, an ethyleneltetrafluoroethylene copolymer (ETFE), a tetrafluoroethylene/propylene copolymer (TFE/P), a tetrafluoroethylene/hexafluoropropylene copolymer (FEP/HFP), a hexafluoropropylene/tetrafluoroethylene/vinylidene copolymer (THV), or a perfluoro(1-butenyl vinyl ether) homocyclopolymer having functionalized polymer-end groups.

### Embodiment 35

The bag of any of Embodiments 32-34, wherein the porous membrane has an average pore size of 10-200 μm, e.g., 20-200 μm, or 30-200 μm, or 75-200 μm, or 10-150 μm, or 20-150 μm or 30-150 μm, or 50-150 μm, or 75-150 μm.

### Embodiment 36

The bag of any of Embodiments 32-35, wherein the porous membrane has a porosity of 10-90% (e.g., 20-70%, or 30-50%).

### Embodiment 37

The bag of any of Embodiments 1-18, comprising
a first porous membrane separating a first lateral portion of the interior compartment from a central portion of the interior compartment;
a second porous membrane separating a second lateral portion of the interior compartment from the central portion of the interior compartment, the central portion containing the plurality of microcarriers; and
a third port formed in an exterior surface of the bag;
   wherein the first port is in fluid communication with the first lateral portion of the interior compartment, the second port is in fluid communication with the second lateral portion of the interior compartment, and the third port is in fluid communication with the central portion of the interior compartment.

### Embodiment 38

The bag of Embodiment 37, wherein the first porous membrane and the second porous membrane each individually comprise a fluoropolymer.

### Embodiment 39

The bag of Embodiment 37, wherein the first porous membrane and the second porous membrane each individually comprise one or more of fluorinated ethylene-propylene (FEP), polytetrafluoroethylene (PTFE), polyvinylidenefluoride (PVDF), tetrafluoroethylene-perfluoro(propyl vinyl ether) (PFA), polyvinylidene difluoride (PVF), polychlorotrifluoroethlylene (PCTFE), a tetrafluoroethylene/hexafluoropropylene/ethylene copolymer (HTE), a chlorotrifluoroethylene/vinylidenefluoride copolymer, a chlorotrifluoroethylene/hexafluoropropylene copolymer, an ethylene/chlorotrifluoroethylene copolymer (ECTFE), an ethylene/trifluoroethylene copolymer, an ethylene/tetrafluoroethylene copolymer (ETFE), a tetrafluoroethylene/propylene copolymer (TFE/P), a tetrafluoroethylene/hexafluoropropylene copolymer (FEP/HFP), a hexafluoropropylene/tetrafluoroethylene/vinylidene copolymer (THV), or a perfluoro(1-butenyl vinyl ether) homocyclopolymer having functionalized polymer-end groups.

### Embodiment 40

The bag of any of Embodiments 37-39, wherein the first porous membrane and the second porous membrane each individually have an average pore size of 50-200 μm (e.g., 50-150 μm, or 75-150 μm).

### Embodiment 41

The bag of any of Embodiments 37-40, wherein the first porous membrane and the second porous membrane each individually have a porosity of 10-90% (e.g., 20-70%, or 30-50%).

### Embodiment 42

A method for cultivating cells, comprising providing anchorage-dependent cells and media together with the microcarriers in the interior compartment the bag of any of Embodiments 1-41, and flowing media through the interior compartment between the first port and the second port.

### Embodiment 43

The method of Embodiment 42, comprising agitating the bag (e.g., by rocking).

### Embodiment 44

The method of any of Embodiments 42-43, further comprising adding, after an incubation period, an additional plurality of microcarriers to the interior compartment of the bag (e.g., through a third port formed in an exterior surface of the bag).

### Embodiment 45

The method of any of Embodiments 42-44, further comprising
separating cultivated cells from the microcarriers; and then
removing the cultivated cells from the bag.

### Embodiment 46

The method for cultivating cells of any of Embodiments 42-45, wherein the bag is a bag according to any of Embodiments 19-31 and wherein media is flowed through the interior compartment by adding media through the second port and through the second liquid-permeable tube and removing media through the first liquid-permeable tube and through the first port.

### Embodiment 47

The method of Embodiment 46, wherein providing the bag comprises adding, through a third port formed in an exterior surface of the bag, the plurality of microcarriers and the media.

### Embodiment 48

The method of Embodiment 46 or Embodiment 47, further comprising
separating cultivated cells from the microcarriers; and then
removing the cultivated cells from interior compartment of the bag, through the first liquid-permeable tube and through the first port.

### Embodiment 49

The method for cultivating cells of any of Embodiments 42-45, wherein the bag is a bag according to any of Embodiments 32-36 and wherein media and anchorage-dependent cells are provided in the second portion of the interior compartment; and wherein media is flowed through the interior compartment by adding media through the second port and removing media through the first port.

### Embodiment 50

The method for cultivating cells of any of Embodiments 42-45, wherein the bag is a bag according to any of Embodiments 37-41 including media and anchorage-dependent cells in the central portion of the interior compartment; and wherein media is flowed through the interior compartment by adding media through the second port and removing media through the first port.

### Embodiment 51

The method of Embodiment 49 or Embodiment 50, wherein the plurality of microcarriers is provided to the interior compartment through the third port.

1. An oxygen-permeable bag comprising
one or more polymer films defining a boundary of an interior compartment of the bag,
each of the one or more polymer films comprising
an inner layer adjacent the interior compartment of the bag, the inner layer comprising a fluoropolymer; and
adhered to the inner layer, an outer layer comprising a polymer;
a first port formed in an exterior surface of the bag and in fluid communication with the interior compartment;
a second port formed in an exterior surface of the bag and in fluid communication with the interior compartment; and
contained in the interior compartment, a plurality of microcarriers.

2. The bag of claim 1, wherein the oxygen-permeable bag is an oxygen-permeable bag comprising
one or more polymer films having edges bonded to form an interior compartment of the bag, each of the one or more polymer films comprising an inner layer adjacent the interior compartment of the bag, the inner layer comprising a fluoropolymer, and adhered to the inner layer, an outer layer comprising a polymer;
a first port formed in an exterior surface of the bag and in fluid communication with the interior compartment;
a second port formed in an exterior surface of the bag and in fluid communication with the interior compartment; and
contained in the interior compartment, a plurality of microcarriers.

3. The bag of claim 1, wherein the inner layer comprises fluorinated ethylene-propylene.

4. The bag of claim 1, wherein the outer layer comprises an elastomer.

5. The bag of claim 1, wherein the outer layer comprises silicone rubber (e.g., selected from high consistency rubber (HCR), fluorosilicone rubber (FSR), liquid silicone rubber (LSR), room temperature vulcanized rubber (RTV), thermoplastic silicone rubber (TPE), platinum-cured silicone rubber, and peroxide-cured silicone rubber).

6. The bag of claim 1, wherein
the inner layer has a thickness of 0.001-0.7 mm (e.g., 0.01-0.1 mm) and comprises substantially (e.g., at least 95 wt. %) fluorinated ethylene-propylene; and
the outer layer has a thickness of 0.01-5 mm (e.g., 0.1-0.5 mm) and comprises substantially (e.g., at least 95 wt. %) silicone rubber.

7. The bag of claim 1, wherein the microcarriers have an average diameter of 100-400 μm (e.g., 110-300 μm, or 120-275 μm).

8. The bag of claim 1, wherein the microcarriers comprise polystyrene, cross-linked dextran, or cellulose.

9. The bag of claim 1, wherein the plurality of microcarriers contained in the interior compartment has a total surface area that is at least 100% (e.g., at least 500%, or at least 1000%) of a total surface area of the interior compartment of the bag.

10. The bag of claim 1, comprising
an undivided interior compartment;
a first liquid-permeable tube extending into and in fluid communication with the interior compartment, the first liquid-permeable tube being operatively coupled to the first port; and
a second liquid-permeable tube extending into and in fluid communication with the interior compartment, the second liquid-permeable tube being operatively coupled to the second port;
wherein the first liquid-permeable tube comprises a first inner support structure defining a central lumen of the tube and a first outer filter layer surrounding the first inner support structure.

11. The bag of claim 1, comprising
an undivided interior compartment;
a first liquid-permeable tube extending into and in fluid communication with the interior compartment of the bag, the first liquid-permeable tube being operatively coupled to the port; and
a second liquid-permeable tube extending into and in fluid communication with the interior compartment of the bag, the second liquid-permeable tube being operatively coupled to the second port;
wherein the first liquid-permeable tube has an average pore size of 50-200 μm (e.g., 50-150 μm, or 75-150 μm).

12. The bag of claim 1, comprising
a porous membrane separating a first portion of the interior compartment from a second portion of the interior compartment, the second portion containing the plurality of microcarriers; and
a third port formed in an exterior surface of the bag;
wherein the first port and second port are each in fluid communication with the first portion of the interior compartment, and the third port is in fluid communication with the second portion of the interior compartment.

13. The bag of claim 1, comprising
a first porous membrane separating a first lateral portion of the interior compartment from a central portion of the interior compartment;
a second porous membrane separating a second lateral portion of the interior compartment from the central portion of the interior compartment, the central portion containing the plurality of microcarriers; and
a third port formed in an exterior surface of the bag;
wherein the first port is in fluid communication with the first lateral portion of the interior compartment, the second port is in fluid communication with the second lateral portion of the interior compartment, and the third port is in fluid communication with the central portion of the interior compartment.

14. A method for cultivating cells, comprising providing anchorage-dependent cells and media together with the microcarriers in the interior compartment the bag of claim 1, and flowing media through the interior compartment between the first port and the second port.

15. The method of claim 14, further comprising
separating cultivated cells from the microcarriers; and then
removing the cultivated cells from the bag.
